(19) United States
(12) Patent Application Publication
(10) Pub. No.: US 2019/0298827 A1
(43) Pub. Date: Oct. 3, 2019

(54) Methods of Treating Multiple Myeloma

(71) Applicant: Janssen Biotech, Inc., Horsham, PA (US)

(72) Inventors: Hong Xie, Spring House, PA (US); Andrew Cakana, Buckinghamshire (GB)

(21) Appl. No.: 16/374,031

(22) Filed: Apr. 3, 2019

Related U.S. Application Data
(60) Provisional application No. 62/651,895, filed on Apr. 3, 2018.

(51) Int. Cl. A61K 39/395; A61K 31/69; A61K 31/198; A61K 31/573; C07K 16/28; A61K 9/00; A61K 47/12; A61K 47/02; A61K 47/26; A61K 47/42; A61K 47/22; A61K 47/20; A61P 35/00

(52) U.S. Cl. CPC A61K 39/3955; A61K 2039/505; A61K 31/198; A61K 31/573; C07K 16/2896; A61K 9/0019; A61K 47/12; A61K 47/02; A61K 47/26; A61K 47/42; A61K 47/22; A61K 47/20; A61K 9/0053; A61K 39/39591; A61P 35/00; C07K 2317/24; A61K 31/69

(57) ABSTRACT

Described herein are methods of treating multiple myeloma with clinically proven safe and effective amounts of an antibody that specifically recognizes CD38 with bortezomib, melphalan, and prednisone. Also described are methods of selling or offering for sale an antibody that specifically recognizes CD38 or pharmaceutical compositions thereof with bortezomib, melphalan, and prednisone.

### CROSS REFERENCE TO RELATED APPLICATIONS

This application claims the benefit of U.S. provisional application Ser. No. 62/651,895 filed 3 Apr. 2018. the entire contents of the aforementioned application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 1, 2019, is named JBI5155USNP1_ST25.txt and is 9 kilobytes in size.

### FIELD OF THE INVENTION

Disclosed herein are clinically proven safe and effective methods of treating multiple myeloma using an antibody that specifically recognizes CD38 with bortezomib, melphalan, and prednisone. Also disclosed are methods of selling or offering for sale an antibody that specifically recognizes CD38 or pharmaceutical compositions thereof with instructions for safely and effectively treating multiple myeloma using the antibody with bortezomib, melphalan, and prednisone.

### BACKGROUND OF THE INVENTION

Multiple myeloma, a malignant disorder of plasma cells, is characterized by uncontrolled and progressive proliferation of a plasma cell clone. Multiple myeloma is estimated to represent 0.8% of all cancers worldwide (Ferlay et al., Int J Cancer. 2015; 136, E359-E386, the disclosure of which is hereby incorporated by reference in its entirety). The incidence of multiple myeloma increases steadily with age, with a median age at diagnosis of approximately 65 to 72 years (Howlader, et al. (Eds.), SEER cancer statistics review, 1975-2014, National Cancer Institute. Bethesda, Md., www_seer.cancer.gov/csr/1975_2014/, based on November 2016 SEER data submission; Merz, et al., Ann Hematol. 2017, 96:987-993; Song, et al., Curr Med Res Opin. 2016; 32(1):95-103; the disclosures of which are hereby incorporated by reference in their entirety), and there is evidence that the incidence of this hematologic malignancy is increasing in developed countries (Becker, Recent Results Cancer Res. 2011, 183:25-35, Cancer Stat Facts: Myeloma. National Cancer Institute Surveillance, Epidemiology, and End Results Program Web site, www_seer. cancer.gov/statfacts/html/mulmy.html.; Velez et al., BMJ Open. 2016; 6(1):e009584; the disclosures of which are hereby incorporated by reference in their entirety) in line with the aging of the population. The annual number of new cases of multiple myeloma diagnosed is estimated to be approximately 30,300 in US (American Cancer Society (ACS). What are the key statistics about multiple myeloma? www_cancer.org/cancer/multiple-myeloma/about/key-statistics.html, the disclosure of which is hereby incorporated by reference in its entirety) and 38,900 overall in Europe (Ferlay 2015, supra).

Multiple myeloma remains an incurable disease. Worldwide, there were an estimated 80,000 deaths due to multiple myeloma based on 2012 data (id.). Approximately 24,300 and 12,600 patients with this disease die annually in Europe and the US, respectively (American Cancer Society 2017, supra; Ferlay 2015, supra).

Patients with newly diagnosed multiple myeloma are typically categorized into two subpopulations defined by their age and suitability for intensive treatment. For patients who are considered fit, an induction regimen followed by high dose chemotherapy and autologous stem cell transplant is considered the frontline treatment standard of care. This therapeutic approach is customarily limited to younger patients. For patients considered ineligible for high dose chemotherapy and autologous stem cell transplant, the frontline treatment approach favors longer, less intensive, less toxic treatments.

Bortezomib (VELCADE®) is an important agent for the treatment of multiple myeloma, and is widely used in combination with an alkylating agent and a steroid in patients with newly diagnosed disease who are considered ineligible for autologous stem cell transplant.

The combination of bortezomib plus melphalan plus prednisone (VMP) is the standard of care therapy in patients ineligible for transplant. The combination of lenalidomide (REVLIMID®) plus dexamethasone (Rd) is also approved for use in this population.

There remains a need for new, clinically proven safe and effective therapeutic options for the frontline setting that can better control the disease and provide deeper, more sustained responses and better long-term outcomes, including maintenance of health-related quality of life.

### SUMMARY OF THE INVENTION

Described herein are methods of treating multiple myeloma comprising, consisting of, or consisting essentially of administering a safe and effective amount of an antibody that specifically recognizes CD38, with bortezomib, melphalan, and prednisone to a human subject who has or is suspected to have multiple myeloma. In some embodiments, the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2 (see Table 5). In further embodiments, the antibody that specifically recognizes CD38 comprises the VH of SEQ ID NO:1 and the VL of SEQ ID NO: 2. In further embodiments, the antibody that specifically recognizes CD38 comprises the HC of SEQ ID NO: 3 and the LC of SEQ ID NO: 4. In further embodiments, the antibody that specifically recognizes CD38 is daratumumab.

In some embodiments, the multiple myeloma is a newly diagnosed multiple myeloma. In certain embodiments, the multiple myeloma is diagnosed according to CRAB criteria, which consider Calcium elevation, Renal insufficiency, Anemia, and Bone marrow abnormalities. In some embodiments, the human subject has detectable M protein in serum or urine. In some embodiments, the human subject has serum free light chain (SFLC) equal or over 100 mg/L and an abnormal serum kappa lambda ration.

In some embodiments, the human subject is ineligible for high-dose chemotherapy. In other embodiments, the human subject is ineligible for autologous stem cell transplant therapy. In still further embodiments, the human subject has received no prior therapy for multiple myeloma.

In some embodiments, administration of a safe and effective amount of the antibody that specifically recognizes CD38, bortezomib, melphalan, and prednisone provides an increase in progression-free survival of the human subject. In other embodiments, administration of a safe and effective amount of the antibody that specifically recognizes CD38, bortezomib, melphalan, and prednisone provides improved overall response rate, improved very good partial response or better rate, improved complete response rate, or improved minimal residual disease rate. In further embodiments, administration of a safe and effective amount of the antibody that specifically recognizes CD38, bortezomib, melphalan, and prednisone provides reduced risk of disease progression or increased time to subsequent anti-myeloma therapy. In some embodiments, administration of a safe and effective amount of the antibody that specifically recognizes CD38, bortezomib, melphalan, and prednisone results in no increase in overall frequency of treatment emergent adverse events.

In some embodiments, the antibody that specifically recognizes CD38 is administered by intravenous infusion; in some embodiments it is administered at a dose of 16 mg/kg. In other embodiments, it is administered once every week for 6 weeks, then once every 3 weeks for an additional 16 doses, then once every 4 weeks thereafter.

In some embodiments, the antibody that specifically recognizes CD38 is administered by subcutaneous injection.

In some embodiments, bortezomib is administered by subcutaneous injection and melphalan and prednisone are administered orally. In other embodiments, bortezomib is administered twice every week in weeks 1, 2, 4, and 5 of a first 6-week cycle, then once every week in weeks 1, 2, 4, and 5 of subsequent 6-week cycles thereafter; and melphalan and prednisone are administered on days 1-4 of each 6-week cycle. In further embodiments, bortezomib is administered at a dose of 1.3 mg/m². In still further embodiments, melphalan is administered at 9 mg/m² and prednisone is administered at 60 mg/m². In still further embodiments, the melphalan and the prednisone are self-administered.

In another aspect, described herein are methods of selling an antibody that specifically recognizes CD38 comprising, consisting of, or consisting essentially of placing the antibody into the stream of commerce wherein said antibody includes a package insert that contains instructions for safe and effective treatment of multiple myeloma using the antibody, bortezomib, melphalan, and prednisone. In certain embodiments, the antibody that specifically recognizes CD38 is daratumumab.

In further aspects, described herein are methods of selling a pharmaceutical composition containing an antibody that specifically recognizes CD38 comprising, consisting of, or consisting essentially of placing such pharmaceutical composition into the stream of commerce wherein said pharmaceutical composition includes a package insert that contains instructions for safe and effective treatment of multiple myeloma using the antibody, bortezomib, melphalan, and prednisone. In certain embodiments, the antibody is daratumumab.

In still further aspects, described herein are methods of offering for sale an antibody that specifically recognizes CD38 comprising, consisting of, or consisting essentially of offering to place the antibody into the stream of commerce wherein said antibody includes a package insert that contains instructions for safe and effective treatment of multiple myeloma using the antibody. In certain embodiments, the antibody is daratumumab.

### BRIEF DESCRIPTION OF THE DRAWINGS

The summary, as well as the following detailed description, is further understood when read in conjunction with the appended drawings. For the purpose of illustrating the disclosed methods, the drawings show exemplary embodiments of the methods; however, the methods are not limited to the specific embodiments disclosed. In the drawings:

FIG. 1 illustrates a schematic overview of the Phase 3 Study described herein. “Dara” refers to daratumumab; “PD” refers to disease progression; “Q4wks” means every 4 weeks; “Q16wks” means every 16 weeks; “T&E” means treatment and evaluation cycle; and “VMP” is a combination therapy using bortezomib+melphalan+prednisone.

FIG. 2 schematically illustrates subject disposition at the clinical cut-off date in the Phase 3 study described herein. In FIG. 2, “D-VMP” is a combination therapy using daratumumab+bortezomib+melphalan+prednisone.

FIG. 3 illustrates a Kaplan-Meier plot for progression-free survival in the intent-to-treat population based on a computer algorithm.

FIG. 4 illustrates a forest plot of subgroup analyses of progression-free survival in the intent-to-treat population based on a computer algorithm. “AST” refers to aspartate aminotransferase; “CI” means confidence interval; “CrCL” refers to creatinine clearance; “ECOG” refers to Eastern Cooperative Oncology group; “EVT” means event; “FISH” means fluorescence in situ hybridization; “ISS” refers to the International Staging System; “MM” refers to multiple myeloma; “NE” means not estimable; and “ULN” means upper limit of normal. Impaired baseline hepatic function includes: mild (total bilirubin≤ULN and AST>ULN) or (ULN<total bilirubin≤1.5×ULN); moderate (1.5×ULN<total bilirubin≤3×ULN); and severe (total bilirubin>3×ULN). High risk cytogenetics is defined as: 1) by FISH testing: t(4; 14), t(14; 16), and 17p deletion; or 2) by Karyotype testing: t(4; 14), 17p deletion. The type of MM subgroup analysis was based on subjects with measurable disease in serum. European Union includes Russian Federation and Turkey (stratified as OTHER at the randomization).

### DETAILED DESCRIPTION OF THE INVENTION

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

### Definitions

The term “safe and effective” refers to an amount and/or dosage of a drug or a combination of drugs that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is evaluated in laboratory, animal and human clinical testing to determine the highest tolerable dose or the optimal dose of the drug or the combination of drugs needed to achieve the desired benefit. Efficacy is evaluated in human clinical testing in Phase 2 or Phase 3 clinical trials and determining whether the drug or the combination of drugs demonstrates a health benefit over a placebo or other intervention Safe and effective drugs or a combination of drugs are granted marketing approval by the FDA for their indicated use.

“Dosage” refers to the information of the amount of the therapeutic to be taken by the subject and the frequency of the number of times the therapeutic is to be taken by the subject.

“Dose” refers to the amount or quantity of the therapeutic to be taken each time.

The term “acceptable” with respect to a formulation, composition or ingredient, as used herein, means having no persistent detrimental effect on the general health of the human subject being treated.

The term “cancer” as used herein refers to an abnormal growth of cells which tend to proliferate in an uncontrolled way and, in some cases, to metastasize (spread) to other areas of a patient's body.

The term “multiple myeloma” as used herein refers to a malignant disorder of plasma cells characterized by uncontrolled and progressive proliferation of a plasma cell clone. The abnormal proliferation of plasma (myeloma) cells causes displacement of the normal bone marrow leading to dysfunction in hematopoietic tissue and destruction of the bone marrow architecture, resulting in progressive morbidity and eventual mortality.

The term “CD38” as used herein refers to cluster of differentiation 38 protein, a glycoprotein expressed on immune cells, including plasma cells, natural killer cells and subpopulations of B and T cells. In some embodiments, the malignant plasma cells of the multiple myeloma express CD38.

An antibody that “specifically recognizes CD38” as used herein refers to an antibody or fragment thereof that immunospecifically binds to CD38. In some embodiments, the antibody that specifically recognizes CD38 is daratumumab.

The term “newly diagnosed” as used herein refers to a human patient who has been diagnosed with but has not yet received treatment for multiple myeloma.

Multiple myeloma is diagnosed according to guidelines provided by the International Myeloma Working Group (IMWG). Subjects with multiple myeloma satisfy CRAB criteria (Calcium elevation, renal insufficiency, anemia and bone abnormalities) (Rajkumar et al., Lancet Oncol 14: e538-48, 2014) and have evidence of measurable secretory disease (measurable M protein in serum or urine; or serum free light chain (SFLC) ≥100 mg/L (involved light chain) and an abnormal serum kappa lambda ratio.

Patients with newly diagnosed multiple myeloma are typically categorized into 2 subpopulations defined by their age and suitability for intensive treatment. In some embodiments, the multiple myeloma treated is a newly diagnosed multiple myeloma.

As used herein, a “frontline” or “firstline” therapy refers to the first treatment of a disease administered to the subject.

As used herein, “high dose chemotherapy” (HDC) and “autologous stem cell transplant” (ASCT) refer to the standard of care for patients with newly diagnosed multiple myeloma who are considered fit. Patients or subjects over the age of 65 years are usually not considered eligible for ASCT due to their frail physical status and presence of comorbidities, which increase the risk of mortality and transplant-related complications (Gay F, Palumbo A. Blood Reviews. 2011; 25:65-73; Mateos, et al. Blood Rev. 2015; 29:387-403; the disclosures of which are hereby incorporated by reference in their entirety). Comorbidities such as renal dysfunction are present at initial diagnosis in a significant proportion of patients with multiple myeloma (12% to 30%) (Song et al. Curr Med Res Opin. 2016; 32(1):95-103; Yong et al. Br J Hematol, 2016; 175(2): 252-264; the disclosures of which are hereby incorporated by reference in their entirety). For patients considered ineligible for HDT and ASCT due to their age, presence of comorbidities, and/or physical status, the treatment approach often favors longer, less intensive, less toxic treatments. In some embodiments of the methods provided herein, the subject treated is ineligible for HDC and ASCT.

The term “progression-free survival (PFS)” means time from initiation of therapy to first evidence of disease progression or death due to any cause, whichever occurs first. For the purpose of the clinical trial described in the example, PFS is defined as the time from randomization of study population to the first documented progressive disease or death due to any cause. In some embodiments, administration of a safe and effective amount and/or dosage of an antibody that specifically recognizes CD38 with bortezomib, melphalan, and prednisone provides an increase in the progression-free survival of a human subject with multiple myeloma. The increase in the progression-free survival refers to an increase in PFS in multiple myeloma (MM) patients treated with daratumumab, bortezomib, melphalan and prednisone relative to PFS in MM patients treated with bortezomib, melphalan, and prednisone only. In some embodiments, the increase in progression-free survival is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, about 36 months, or greater than 36 months.

The terms “partial response” (PR), “very good partial response” (VGPR), “complete response” (CR), “stringent complete response” (sCR), as used herein take their customary meanings as will be understood by a person skilled in the art of designing, conducting, or reviewing clinical trials. International Uniform Response Criterial Consensus Recommendations can be used to assess response. The term “overall response rate” (ORR) as used herein amounts to the sum of the rates of PR+VGPR+CR+sCR.

The term “Minimal residual disease (MRD)” as used herein refers to a small number of multiple myeloma cells that remain in the patient after treatment and/or during remission. “MRD negative” refers to a ratio of 1:10×10⁵ or less clonal multiple myeloma cells in a bone marrow aspirate sample from the subject.

The term “time to subsequent anti-myeloma therapy” as used herein refers to the time from the initiation of therapy to documentation of administration of a new anti-myeloma therapy to the subject. In some embodiments, administration of a safe and effective amount of an antibody that specifically recognizes CD38 with bortezomib, melphalan, and prednisone provides improved anti-myeloma activity as measured by time to subsequent anti-myeloma therapy.

The term “treatment emergent adverse events” (TEAE) as used herein takes its customary meaning as will be understood by a person skilled in the art of designing, conducting, or reviewing clinical trials.

The terms “co-administration,” “administration with,” “administration in combination with,” or the like, as used herein, encompass administration of the selected therapeutic agents to a single patient, and are intended to include treatment regimens in which the agents are administered by the same or different route of administration or at the same or different time.

The terms “treat,” “treating,” “treated,” and “treatment” refer to therapeutic intervention of a patient afflicted with a pathological condition and refers to an effect that alleviates the condition, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “progressive disease” refers to increase of 25% from lowest response value in any one of the following:
1. Serum M-component (absolute increase must be ≥0.5 g/dL)
2. Urine M-component (absolute increase must be ≥200 mg/24 hours)
3. Only in subjects without measurable serum and urine M-protein levels: the difference between involved and uninvolved FLC levels (absolute increase must be >10 mg/dL)
4. Only in subjects without measurable serum and urine M-protein levels and without measurable disease by FLC levels, bone marrow PC percentage (absolute percentage must be ≥10%)
5. Bone marrow plasma cell percentage: the absolute percentage must be □10%
6. Definite development of new bone lesions or soft tissue plasmacytomas or definite increase in the size of existing bone lesions or soft tissue plasmacytomas
7. Development of hypercalcemia (corrected serum calcium >11.5 mg/dL) that can be attributed solely to the PC proliferative disorder

The term “overall survival (OS)” is defined as the time from initiation of therapy to the date of death due to any cause. For the purpose of the clinical trial described in the example, OS is defined as the time from randomization of study population to the date of the patient's death. In some embodiments, administration of a safe and effective amount and/or dosage of an antibody that specifically recognizes CD38 with bortezomib, melphalan, and prednisone provides an increase in the overall survival of a human subject with multiple myeloma. The increase in the overall survival refers to an increase in the overall survival in multiple myeloma (MM) patients treated with daratumumab, bortezomib, melphalan and prednisone relative to the overall survival in MM patients treated with bortezomib, melphalan, and prednisone only.

The term “progression-free survival with the first subsequent therapy” (PFS2) is defined as the time from initiation of therapy to progression on the next line of therapy or death, whichever comes first. In some embodiments, administration of a safe and effective amount and/or dosage of an antibody that specifically recognizes CD38 with bortezomib, melphalan, and prednisone provides an increase in the PFS2 of a human subject with multiple myeloma. The increase in the PFS2 refers to an increase in the PFS2 in multiple myeloma (MM) patients treated with daratumumab, bortezomib, melphalan and prednisone relative to the PFS2 in MM patients treated with bortezomib, melphalan, and prednisone only.

The terms “kit” and “article of manufacture” are used as synonyms.

The term “subject” and “patient” and “human” are used interchangeably.

The term “antibody” includes immunoglobulin molecules including monoclonal antibodies including human, humanized and chimeric monoclonal antibodies. Full-length antibody molecules are comprised of two heavy chains (HC) and two light chains (LC) inter-connected by disulfide bonds. Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDRs), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDRs) are “antigen binding sites” in an antibody. CDRs may be defined based on sequence variability (Wu and Kabat, (1970) J Exp Med 132:211-50; Kabat et al., Sequences of Proteins of Immunological Interest, 5th Ed. Public Health Service, National Institutes of Health, Bethesda, Md., 1991; hereby incorporated by reference for such disclosure) or based on alternative delineations (see Lefranc et al., (2003) Dev. Comparat. Immunol. 27:55-77, hereby incorporated by reference for such disclosure). The International ImMunoGeneTics (IMGT) database (http://www_imgt_org) provides a standardized numbering and definition of antigen-binding sites.

Routes of Administration and Pharmaceutical Compositions

Therapeutic agents described herein are administered in any suitable manner or suitable formulation. Suitable routes of administration of the therapeutic agents include, but are not limited to, oral and parenteral (e.g., intravenous, subcutaneous, intramuscular). All formulations are in dosages suitable for administration to a human. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, administration of a safe and effective amount of an antibody that specifically recognizes CD38 with bortezomib, melphalan, and prednisone results in no more than a grade 2 adverse event. In other embodiments, administration of a safe and effective amount of antibody that specifically recognizes CD38 with bortezomib, melphalan, and prednisone results in no more than a grade 3 adverse event. In other embodiments, administration of a safe and effective amount of antibody that specifically recognizes CD38 with bortezomib, melphalan, and prednisone results in no more than a grade 4 adverse event.

In some embodiments, the antibody that specifically recognizes CD38 is administered intravenously. In other embodiments, the antibody that specifically recognizes CD38 is administered subcutaneously.

In some embodiments, the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2 as defined by Kabat (supra) (see Table 5). In some embodiments, the antibody that specifically recognizes CD38 comprises the VH of SEQ ID NO: 1 and the VL of SEQ ID NO: 2. In some embodiments, the antibody that specifically recognizes CD38 comprises the heavy chain (HC) of SEQ ID NO: 3 and the light chain (LC) of SEQ ID NO: 4. In some embodiments, the antibody that specifically recognizes CD38 is daratumumab.

In some embodiments, the antibody that specifically recognizes CD38 is administered in a pharmaceutical composition comprising about 20 mg/ml of the antibody, about 25 mM acetate, about 60 mM sodium chloride, about 140 mM mannitol and about 0.04% (w/v) PS 20, pH 5.5

In some embodiments, bortezomib is administered subcutaneously.

To prepare the pharmaceutical compositions containing an antibody that specifically recognizes CD38 of this invention and/or bortezomib, one or more active pharmaceutical ingredients is intimately admixed with a pharmaceutical carrier or solvent according to conventional pharmaceutical compounding techniques, which carrier or solvent may take a wide variety of forms depending of the form of preparation desired for administration (e.g. intravenous, intraperitoneal, intramuscular, or other). Suitable pharmaceutically acceptable carriers and solvents are well known in the art. Descriptions of some of these pharmaceutically acceptable carriers may be found in The Handbook of Pharmaceutical Excipients, published by the American Pharmaceutical Association and the Pharmaceutical Society of Great Britain. Preferably, these compositions are in unit dosage forms.

In some embodiments, a single unit dosage of daratumumab composition comprises, consists of, or consists essentially of about 16 mg/kg body weight of daratumumab. In some embodiments, a single unit dosage of a bortezomib composition comprises, consists of, or consists essentially of about 1.3 mg/m² body surface area of bortezomib.

In some embodiments, melphalan and prednisone are administered orally. In further embodiments, melphalan and prednisone are administered in solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques. Preferably these compositions are in unit dosage forms from such as tablets, pills, capsules, dry powders for reconstitution or inhalation, granules, lozenges, sterile solutions or suspensions, metered aerosol or liquid sprays, drops, or suppositories for administration by oral, intranasal, sublingual, intraocular, transdermal, rectal, vaginal, dry powder inhaler or other inhalation or insufflation means.

These compositions and formulations are manufactured by conventional techniques. For preparing solid pharmaceutical compositions such as tablets, the principal active ingredient is mixed with a pharmaceutical carrier, e.g., conventional tableting ingredients such as diluents, binders, adhesives, disintegrants, lubricants, antiadherents, and gildants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL silica available from Cabot, SYLOID silica available from W.R. Grace/Davison, and AEROSIL silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

In certain embodiments, inactive ingredients of a core tablet are: colloidal anhydrous silica, croscarmellose sodium, hydroxypropyl methylcellulose-acetate succinate, magnesium stearate, microcrystalline cellulose, and silicified microcrystalline cellulose. In other embodiments, the tablets are finished with a film-coating consisting of the following excipients: iron oxide black, iron oxide yellow, polyethylene glycol, polyvinyl alcohol, talc, and titanium dioxide

In some embodiments, a single unit dosage of a melphalan composition comprises, consists of, or consists essentially of about 9 mg/m² of melphalan. In some embodiments, a single unit dosage of a prednisone composition comprises, consists of, or consists essentially of about 60 mg/m² of prednisone.

All formulations for oral administration are in dosage form suitable for such administration.

Methods of Dosing and Treatment Regimens

In one aspect, described herein are methods of treating human subjects with newly diagnosed multiple myeloma who are ineligible for ASCT comprising, consisting of, or consisting essentially of administering a safe and effective amount of an antibody that specifically recognizes CD38 with bortezomib, melphalan, and prednisone to the human subject. In some embodiments, 16 mg/kg of the antibody that specifically recognizes CD38 is administered by intravenous (IV) infusion initially once every week for 6 weeks (6 doses; Cycle 1); then once every 3 weeks for an additional 16 doses (Cycles 2-9); then once every 4 weeks thereafter. In some embodiments, about 1,200 mg to about 1,800 mg of the antibody that specifically recognizes CD38 is administered by subcutaneous injection in a formulation of about 20 mg/mL of the antibody, about 25 mM acetic acid, about 60 mM sodium chloride, about 140 mM mannitol and about 0.04% w/v polysorbate-20 (PS-20); at pH 5.5 with about 30,000 U to 45,000 of recombinant human hyaluronidase (rhPH20) in 10 mM L-Histidine, 130 mM NaCl, 10 mM L-Methionine and 0.02% Polysorbate 80, pH 6.5. In some embodiments, about 1,800 mg of the antibody that specifically recognizes CD38 is administered by subcutaneous injection in a formulation of about 20 mg/mL of the antibody, about 25 mM acetic acid, about 60 mM sodium chloride, about 140 mM mannitol and about 0.04% w/v polysorbate-20 (PS-20); at pH 5.5 with about 45,000 of recombinant human hyaluronidase (rhPH20) in 10 mM L-Histidine, 130 mM NaCl, 10 mM L-Methionine and 0.02% Polysorbate 80, pH 6.5.

In some embodiments, about 1,200 mg to about 5,000 mg of the antibody that specifically recognizes CD38 is administered by subcutaneous injection in a pharmaceutical composition comprising rhPH20 in an amount of from about 30,000 U to about 45,000 U, histidine at a concentration of from about 5 mM to about 15 mM, sorbitol at a concentration of from about 100 mM to about 300 mM, PS-20 at a concentration of from about 0.01% w/v to about 0.04% w/v; and methionine at a concentration of from about 1 mg/mL to about 2 mg/mL, at a pH of about 5.5.

In some embodiments, about 1,200 mg to about 5,000 mg of the antibody that specifically recognizes CD38 is administered by subcutaneous injection in a pharmaceutical composition comprising about 120 mg/mL of the antibody, about 10 mM Histidine, about 300 mM Sorbitol, about 1 mg/mL methionine, about 0.04% Polysorbate 20, and about 2000 U/ml rHuPH20, pH 5.6.

In some embodiments, the antibody that specifically recognizes CD38 is administered by subcutaneous injection once every week for 8 weeks, then once every 2 weeks for an additional 16 weeks, then once every 4 weeks thereafter.

In some embodiments, the antibody that specifically recognizes CD38 is daratumumab.

In another embodiment, 1.3 mg/m² of bortezomib is administered by subcutaneous injection twice every week in weeks 1, 2, 4, and 5 of a first 6-week cycle, then once every week in weeks 1, 2, 4, and 5 of subsequent 6-week cycles thereafter; and melphalan and prednisone are administered orally on days 1-4 of each 6-week cycle. In some embodiments, melphalan is administered at a dose of 9 mg/m². In some embodiments, prednisone is administered at 60 mg/m².

In certain embodiments wherein improvement in the status of the disease or condition in the human is not observed, the dose of antibody that specifically recognizes CD38 is increased. In some embodiments, a once-a-day dosing schedule is changed to a twice-a-day dosing schedule. In some embodiments, a three times daily dosing schedule is employed to increase the amount of antibody that specifically recognizes CD38 that is administered.

In some embodiments, the amount of antibody that specifically recognizes CD38 that is administered to the human varies depending upon factors such as, but not limited to, condition and severity of the disease or condition, and the identity (e.g., age, weight, comorbities) of the human, and the particular additional therapeutic agents that are administered (if applicable).

In further embodiments, a human subject having newly diagnosed multiple myeloma has received at least one prior therapy for the treatment of cancer. In still further embodiments, human subject having newly diagnosed multiple myeloma is cancer treatment naïve.

Kits/Articles of Manufacture

For use in the methods described herein, kits and articles of manufacture are also described. Such kits include a package or container that is compartmentalized to receive one or more doses of the pharmaceutical compositions disclosed herein. Suitable containers include, for example, bottles. In one embodiment, the containers are formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert.

In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more doses containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

Methods of Sale

In another aspect, described herein are methods of selling an antibody that specifically recognizes CD38 comprising, consisting of, or consisting essentially of placing the antibody that specifically recognizes CD38 into the stream of commerce wherein said antibody that specifically recognizes CD38 includes a package insert that contains instructions for safe and effective treatment of human patients with newly diagnosed multiple myeloma who are ineligible for ASCT using the antibody that specifically recognizes CD38, bortezomib, melphalan, and prednisone. In some embodiments, the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2. In some embodiments, the antibody that specifically recognizes CD38 comprises the VH of SEQ ID NO:1 and the VL of SEQ ID NO: 2. In some embodiments, the antibody that specifically recognizes CD38 comprises the HC of SEQ ID NO: 3 and the LC of SEQ ID NO: 4. In some embodiments, the antibody that specifically recognizes CD38 is daratumumab.

In further aspects, described herein are methods of selling a pharmaceutical composition containing antibody that specifically recognizes CD38 comprising, consisting of, or consisting essentially of placing such pharmaceutical composition into the stream of commerce wherein such pharmaceutical composition includes a package insert that contains instructions for safe and effective treatment of human patients with newly diagnosed multiple myeloma who are ineligible for ASCT using antibody that specifically recognizes CD38, bortezomib, melphalan, and prednisone. In some embodiments, the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2. In some embodiments, the antibody that specifically recognizes CD38 comprises the VH of SEQ ID NO:1 and the VL of SEQ ID NO: 2. In some embodiments, the antibody that specifically recognizes CD38 comprises the HC of SEQ ID NO: 3 and the LC of SEQ ID NO: 4. In some embodiments, the antibody that specifically recognizes CD38 is daratumumab.

In still further aspects, described herein are methods of offering for sale antibody that specifically recognizes CD38 comprising, consisting of, or consisting essentially of offering to place the antibody that specifically recognizes CD38 into the stream of commerce wherein said antibody that specifically recognizes CD38 includes a package insert that contains instructions for safe and effective treatment of patients with newly diagnosed multiple myeloma who are ineligible for ASCT using the antibody that specifically recognizes CD38, bortezomib, melphalan, and prednisone. In some embodiments, the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2. In some embodiments, the antibody that specifically recognizes CD38 comprises the VH of SEQ ID NO:1 and the VL of SEQ ID NO: 2. In some embodiments, the antibody that specifically recognizes CD38 comprises the HC of SEQ ID NO: 3 and the LC of SEQ ID NO: 4. In some embodiments, the antibody that specifically recognizes CD38 is daratumumab.

### EXAMPLES

These examples are provided for illustrative purposes only and not to limit the scope of the claims provided herein.

Phase 1b Study

A Phase 1b, multicenter, non-randomized, open-label study was undertaken to provide initial information on the safety and tolerability of daratumumab when administered in combination with various background regimens for multiple myeloma. Results are presented in this disclosure for the cohort of 12 subjects with newly diagnosed multiple myeloma ineligible for autologous stem cell transplant (ASCT) who received the combination of daratumumab with bortezomib (VELCADE)+melphalan+prednisone (D-VMP).

The first subject in this cohort started treatment with D-VMP. Subjects in the D-VMP cohort of the Phase 1b Study received treatment for a maximum of 9 cycles. Long-term follow-up for this cohort continued until death, loss to follow up, withdrawal of consent, or study end, whichever occurred first. The primary objective for the Phase 1b Study was to evaluate safety and tolerability of the combination regimen. Efficacy assessments (primary efficacy endpoint, overall response rate (ORR)) were performed per International Myeloma Working Group (IMWG) guidelines (Kumar S, et al. Lancet Oncol. 2016; 17(8):e328-e346, hereby incorporated by reference in its entirety), and a computerized, validated algorithm as described above was utilized for determining response based on IMWG criteria.

The median follow-up for the 12 subjects in the D-VMP cohort was 33 months, and 10 (83%) subjects completed all 9 cycles of study treatment.

Phase 3 Study

Phase 3 Study Design

A multicenter, Phase 3, randomized, open-label, parallel-group study compared D-VMP with VMP in subjects with newly diagnosed multiple myeloma who are ineligible for high-dose chemotherapy (HDT) with ASCT. The VMP regimen used the approved doses for bortezomib (1.3 mg/m²), melphalan (9 mg/m²), and prednisone (60 mg/m²), the approved number of cycles (9 cycles), and the standard approved cycle length (6 weeks), while twice-weekly bortezomib dosing was reduced from the first 4 cycles to the first cycle alone to improve tolerability in accordance with the above guidelines and standard clinical practice. Twice-weekly bortezomib dosing in the first cycle was retained in both treatment groups to rapidly reduce plasma cell tumor burden at the beginning of treatment.

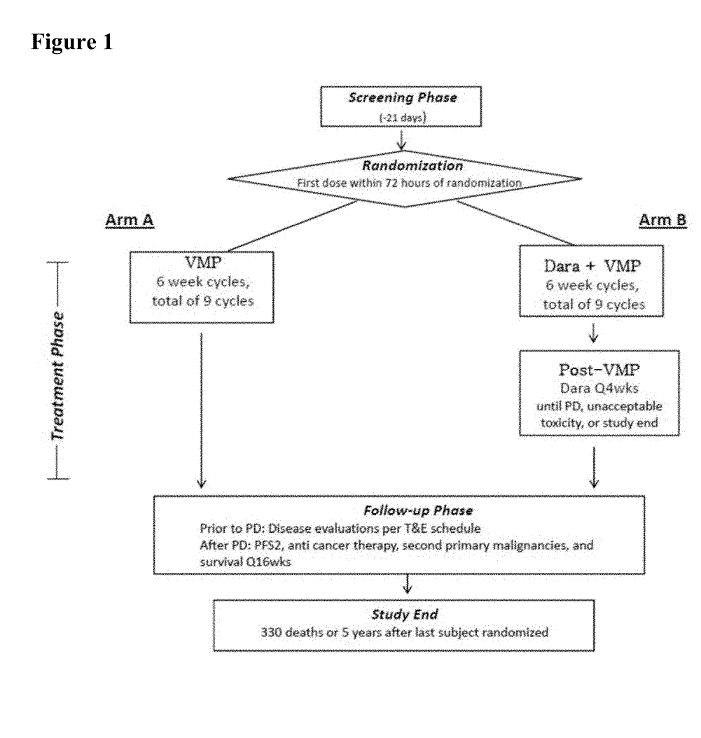

The study consisted of a Screening phase, Treatment phase (from Cycle 1, Day 1 until study treatment discontinuation), and Follow-up phase (see FIG. 1 Error! Reference source not found.). All randomized subjects received VELCADE® (bortezomib) by subcutaneous (SC) injection twice-weekly (Weeks 1, 2, 4, and 5) in Cycle 1 (1 cycle=6 weeks) followed by once weekly (Weeks 1, 2, 4, and 5) in Cycles 2-9. Melphalan per oral at 9 mg/m² and prednisone per oral at 60 mg/m² were to be self-administered on Day 1-4 of each bortezomib cycle.

Subjects randomized to the VMP group were treated for 9 cycles and then entered the Follow-up observation phase. Subjects assigned to the D-VMP group received daratumumab (16 mg/kg) administered by intravenous (IV) infusion initially once every week for 6 weeks (6 doses; Cycle 1); then once every 3 weeks for an additional 16 doses (Cycles 2-9); then once every 4 weeks thereafter (post-VMP Treatment Phase) until documented progression, unacceptable toxicity, or study end.

Subjects entered the Follow-up phase after completion of the VMP cycles in the VMP group or upon discontinuation of daratumumab treatment in the D-VMP group. During the Follow-up phase, subjects who discontinued before disease progression continued to have disease evaluations until confirmed progression, subsequent anticancer treatment, death, withdrawal of consent, lost to follow-up, or study end, whichever occurred first.

The objectives of the Phase 3 Study were to compare the efficacy of daratumumab combined with VMP, a recognized standard of care, to VMP alone; to determine whether the daratumumab-containing regimen improves clinical outcomes in subjects with newly diagnosed multiple myeloma who are ineligible for HDT with ASCT; and to assess the safety and tolerability of daratumumab when administered in combination with VMP. Randomization was used to minimize bias in the assignment of subjects to treatment groups, to increase the likelihood that known and unknown subject attributes (e.g., demographic and baseline characteristics) were evenly balanced across treatment groups, and to enhance the validity of statistical comparisons across treatment groups. To ensure the study population was representative and balanced, randomization was stratified by International Staging System (ISS) category (I, II, or III) and age (<75 vs ≥75) at Screening, and by region (Europe vs Other). Within each stratum, subjects were randomized using an equal allocation ratio of 1:1.

Subjects in the Phase 3 Study were diagnosed with multiple myeloma as defined by the IMWG guidelines (Rajkumar S V, et al. 2011; 117:4691-4695, hereby incorporated by reference in its entirety). Specifically, all subjects had documented multiple myeloma satisfying CRAB criteria (calcium elevation, renal insufficiency, anemia, and bone abnormalities), and had evidence of measurable secretory disease (as determined by a central laboratory). Enrollment was limited to subjects with newly diagnosed disease who had not had any exposure to prior therapy for multiple myeloma and who were not considered candidates for HDT with ASCT due to being ≥65 years of age or having an important comorbid condition(s) likely to negatively impact the tolerability of transplantation. Subjects with a poor performance status (ie, Eastern Cooperative Oncology Group (ECOG) Performance Status Score of 3 or worse) were excluded mainly for safety reasons, as this population of patients generally has a greater risk for toxicity. Also excluded were subjects with peripheral neuropathy or Grade 2 or higher neuropathic pain as neuropathy is a known toxicity associated with bortezomib.

Daratumumab was administered at 16 mg/kg as per the DARZALEX® (daratumumab) prescribing information, but the dosing schedule was modified to match the 6-week cycle schedule for VMP. Daratumumab administration continued until disease progression, unacceptable toxicity, or study end.

A central laboratory was used for disease evaluations (quantitative immunoglobulin, M-protein, and serum free light chain determinations, immunofixation measurements in serum and 24-hour urine).

To increase objectivity, progression-free survival (PFS), the primary endpoint, was determined by the use of a validated computer algorithm that combines laboratory results (eg, monoclonal protein level) and applicable imaging, and generates the outcome according to IMWG criteria. Further, sensitivity analyses of PFS were performed, including those using investigator-determined response.

The study utilized an Independent Data Monitoring Committee (IDMC) and incorporated 2 preplanned interim analyses. The first interim analysis was planned early during the study to assess the safety of continued administration with the study treatment regimens in this newly diagnosed target population. No changes in study conduct were recommended by the IDMC based on the first interim analysis for safety. The objective of the second interim analysis was to evaluate cumulative interim safety and efficacy data and was planned after approximately 216 PFS (progression or death) events had occurred (representing ˜60% of the total planned events).

Following the review of data from the second interim analysis, the IDMC recommended immediately unblinding the study as the pre-specified statistical boundary for PFS was crossed. The study met the primary objective of showing that the combination of daratumumab with VMP improves PFS compared with VMP alone, which was further supported by statistically significant improvements in the key secondary efficacy endpoints of overall response rate (ORR,) very good partial response (VGPR) or better rate, complete response (CR) or better rate, and minimal residual disease (MRD) negativity rate.

Critical Aspects of Phase 3 Study Endpoints

In agreement with Food and Drug Administration and Committee for Medicinal Product for Human Use feedback, the primary endpoint was PFS (defined as the duration from the date of randomization to either progressive disease [based on IMWG response criteria (Dune B G, et al. 2006; 20:1467-1473. Corrigenda/Erratum in: Leukemia. 2007; 21:1134-1135; Rajkumar S V, et al. Blood. 2011; 117:4691-4695; hereby incorporated by reference in their entirety)] or death, whichever occurred first).

Key secondary endpoints included ORR (PR+VGPR+CR+stringent CR), VGPR or better rate (VGPR+CR+stringent CR), CR or better rate (CR+stringent CR), MRD negativity rate, and overall survival (OS). All response-related secondary endpoints were derived using the validated computer algorithm as their primary analyses. Safety and tolerability were also included as key secondary objectives.

Detection of MRD involves testing bone marrow aspirate samples using ultrasensitive methods to confirm the absence of residual myeloma cells after treatment. Here, next generation sequencing (NGS) of immunoglobulin heavy and light chains was utilized. The updated, analytically validated version of the clonoSEQ® Assay (Version 2) by Adaptive Biotechnologies was used for the detection, quantification and analysis of MRD. Calibration success rates using the updated clonoSEQ Assay for testing of multiple myeloma samples were ≥90%.

Phase 3 Study Statistical Methods

The primary hypothesis for the study was tested at the 0.05 2-sided significance level (overall). The exact significance level at the second interim analysis was determined by the observed number of events per the O'Brien-Fleming alpha spending function. With 231 PFS events observed at the second interim analysis (64% of planned 360 total events), the alpha to be spent was 0.0103 (2-sided). An observed 2-sided p-value smaller than this significance level establishes the superiority of D-VMP versus VMP.

### Example 1. Clinical Pharmacology

Clinical pharmacology information provided herein focuses on data from subjects with evaluable pharmacokinetic data treated with D-VMP in the Phase 3 Study (n=342), and supportive information derived from the Phase 1b Study in the cohort of subjects treated with D-VMP having evaluable pharmacokinetic data (n=11). In addition, an updated population pharmacokinetics (PPK) analysis including data from the Phase 3 and Phase 1b Studies and exposure-response (efficacy and safety) analyses including data from Phase 3 Study were conducted.

1-1 Pharmacokinetics

The absorption, distribution, metabolism and excretion characteristics of daratumumab are based on analysis of full pharmacokinetic profiles available for monotherapy dosing. In general, daratumumab serum concentrations in the Phase 3 Study appeared comparable to those observed in prior studies, and the combination of daratumumab with background VMP therapy does not appear to alter the pharmacokinetic profile of daratumumab. Analysis of daratumumab and bortezomib concentrations in the Phase 1b Study also indicated a lack of clinically relevant drug-drug interaction between these molecules.

In the Phase 3 Study and the Phase 1b Study, the mean end-of-infusion concentrations following the first dose of daratumumab 16 mg/kg in subjects receiving the D-VMP combination (266.7 and 332.2 μg/mL, respectively) were comparable to each other and similar to the mean end-of-infusion concentration following the first dose of daratumumab monotherapy (312.5 μg/mL) in a Phase 2 study in relapsed or refractory multiple myeloma. Moderate inter-subject variability for daratumumab exposure was observed among subjects evaluated for pharmacokinetics in the Phase 3 Study.

When co-administered with VMP, daratumumab serum concentrations accumulated after weekly dosing and then decreased slightly during subsequent less frequent dosing periods, in line with data from previous monotherapy studies. In the Phase 1b Study, accumulation of daratumumab after 6 weekly doses (ie, through Cycle 2 Day 1, corresponding to start of every 3-week dosing) resulted in a 2.8-fold increase in daratumumab peak concentration when compared with the first dose. These results are similar to the 2.9-fold increase in daratumumab seen at the end of 8 weekly doses of daratumumab monotherapy in the Phase 2 Study in relapsed or refractory multiple myeloma.

The daratumumab dosing schedule for use in combination with VMP in the Phase 1b and Phase 3 studies (16 mg/kg by IV infusion weekly for 6 weeks [6 doses; Cycle 1] followed by once every 3 weeks for an additional 16 doses [Cycles 2-9]) was slightly modified relative to the previously approved schedules to adapt daratumumab administration to information included in the bortezomib label. Compared with previous studies which began with weekly dosing of daratumumab for 8 to 9 weeks, the D-VMP regimen for newly diagnosed multiple myeloma begins with weekly dosing of daratumumab for 6 weeks. Despite the different schedules, the mean maximum trough concentration of daratumumab after 6 weekly doses in combination with VMP in the Phase 1b Study (588.0 μg/mL) was consistent with the mean maximum trough concentration after 8 weekly doses in the monotherapy Phase 2 Study in relapsed or refractory multiple myeloma (573.5 μg/mL). The maximum trough concentration is defined as the daratumumab effective concentration based on exposure-ORR analyses from the Phase 3 and previous studies. Based on PPK analysis, target saturation >90% is maintained at trough daratumumab concentrations in the majority (>99%) of subjects from the Phase 3 Study during the every 4-week dose regimen.

1-2 Drug-Drug Interactions

No interactions of daratumumab and small molecule drugs such as bortezomib, melphalan, and prednisone are expected as there is no overlapping pathway of elimination. Analysis of daratumumab and bortezomib concentrations from the Phase 1b study indicated a lack of clinically relevant drug-drug interaction between these molecules.

1-3 Population Pharmacokinetic (PPK) Analysis

The PPK was based on 1,635 pharmacokinetic samples from 352 evaluable subjects treated with D-VMP (daratumumab at 16 mg/kg) in the Phase 3 Study and the D-VMP arm of the Phase 1b Study. The observed concentration time data of daratumumab were well described by a 2-compartment PPK model with parallel linear and nonlinear Michaelis-Menten eliminations, consistent with the PPK modeling for previous monotherapy and combination therapy studies. Due to the lack of overlapping clearance mechanism for daratumumab and co-administered small-molecule combination therapies, no direct effect of the combination therapies on the pharmacokinetics of daratumumab is expected. The estimated clearance (CL) value was similar to the CL of non-specific endogenous IgG reported in the literature, and the estimated V₁ value approached plasma volume.

The model-derived half-life associated with the CL of daratumumab was approximately 22.1±9.7 (mean±SD) days, comparable to the half-life of 18±9 (mean±SD) days derived from monotherapy study data and the half-life of 23.3±11.8 (mean±SD) days derived from previous combination therapy study data.

Similar to what was observed in previous monotherapy and combination therapy studies, apparent steady state seemed to be reached at approximately 5 months into the once every 4-week dosing period. The ratio of the steady-state peak concentration after every 4-week dosing and the peak concentration after the first dose was 2.06±0.61 (mean±SD).

Overall, the pharmacokinetics of daratumumab and the effects of the investigated intrinsic and extrinsic factors were similar following the D-VMP dose regimen and the approved dose regimens for daratumumab in previous monotherapy and combination therapy studies. None of the investigated intrinsic and extrinsic factors (ie, age, sex, race, region, renal impairment, hepatic impairment, baseline albumin, ECOG, and type of myeloma) had clinically important effects on the exposure to daratumumab as all the covariate effects were within approximately 20%.

1-3-1 Exposure-Response Relationship

Exposure-response relationships were investigated based on the Phase 3 Study data. Since all subjects in the D-VMP group received the recommended daratumumab dose of 16 mg/kg, there was limited exposure variation and, therefore, only exploratory and graphic exposure-response analyses were performed for selected efficacy endpoints and adverse events (AEs).

The daratumumab dose regimen for the D-VMP combination was effective for patients with newly diagnosed multiple myeloma based on exploratory exposure-response analyses. The relative risk of disease progression/death was markedly reduced with increasing exposure. The maximum drug effect on PFS was attained for the majority of the subjects with an acceptable safety profile following the D-VMP dose regimen.

Subjects in the D-VMP group benefited from treatment with daratumumab as observed by a lower relative risk of disease progression or death across the studied concentration range compared to subjects in the VMP group.

No apparent relationship within the studied concentration range was identified between drug exposure (end-of-infusion concentration after the first infusion for infusion-related reaction and predicted maximal end-of-infusion concentration for other adverse events) and infusion-related reaction (IRR,) thrombocytopenia, anemia, neutropenia, lymphopenia, and infections.

PPK and exposure-response analyses demonstrated similar pharmacokinetics for daratumumab (eg, similar maximal trough concentrations and effects of the intrinsic and extrinsic factors) following the D-VMP dose regimen and previously approved dose regimens for monotherapy and other combination therapies. Target saturation >90% was maintained at trough concentrations in the majority (>99%) of the subjects following the 16 mg/kg every 4-weeks dose regimen (ie, at steady state) for the dose schedule. Consistent with the findings based on the monotherapy data, maximum clinical benefit on efficacy was attained for the majority of the subjects at the recommended dose (16 mg/kg) with an acceptable safety profile.

1-3-2 Immunogenicity

In subjects treated with D-VMP combination therapy, none of the 119 subjects having samples appropriate for immunogenicity evaluation (defined as at least 1 post-dose sample) were positive for anti-daratumumab antibodies (113 subjects in the Phase 3 Study and 6 subjects in the D-VMP cohort of the Phase 1b Study). These results are consistent with a low risk of immunogenicity with daratumumab reported in combination studies involving subjects with relapsed or refractory multiple myeloma.

### Example 2. Efficacy

For the purpose of the present disclosure, the efficacy of daratumumab in combination with VMP in patients with newly diagnosed multiple myeloma considered ineligible for transplant is primarily based on results from the Phase 3 Study. Efficacy analyses were primarily based on the intent-to-treat (ITT) analysis population, comprised of the 706 subjects who were randomly assigned to study treatment with D-VMP (350 subjects) or VMP (356 subjects). At the time of clinical cut-off, the median overall follow-up was 16.5 months.

2-1. Subject Disposition

As of the clinical cut-off date, 706 subjects from 162 centers in 25 countries were randomized into the Phase 3 Study.

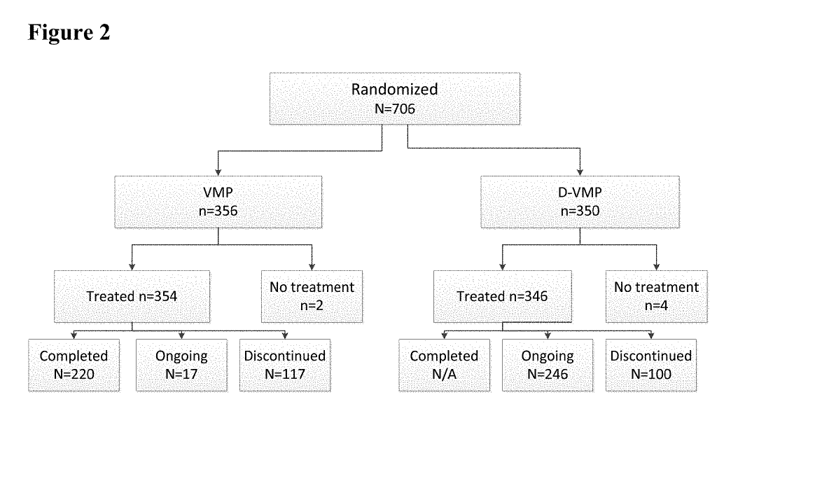

An overview of the subject disposition for the Phase 3 Study is shown in FIG. 2. At the time of the clinical cut-off, 80% of subjects in the D-VMP group and 62% of subjects in the VMP group had completed all 9 protocol-specified cycles of VMP treatment. During the first 9 cycles, 19% of subjects in the D-VMP group and 33% of subjects in the VMP group discontinued treatment, with a lower percentage of subjects in the D-VMP group compared with the VMP group discontinuing treatment due to disease progression (7% vs 13%) or adverse events (AEs) (5% vs 9%), and a similar percentage discontinuing due to death (3% vs 2%).

The majority (71%) of subjects in the D-VMP remained on study treatment as of the clinical cut-off date, with most of these continuing on daratumumab treatment alone (ie, Cycles 10+). Five percent (5%) of subjects in the VMP group remained on study treatment at the time of the clinical cut-off.

2-2 Population Studied

Baseline demographics for the efficacy population in the Phase 3 Study were well-balanced between the D-VMP and VMP groups. The Phase 3 study population was generally representative of the population with newly diagnosed multiple myeloma who are not eligible for transplant, with the 706 randomized subjects having a median age of 71 years (range: 40-93) and 30% of subjects being ≥75 years. The majority of subjects were white (85%) and female (54%); the percentage of subjects with an ECOG performance status score of 0, 1, or 2 at baseline was 25%, 50%, and 25%, respectively.

Disease characteristics at baseline were also generally well-balanced between the 2 treatment groups. The median time from initial multiple myeloma diagnosis to randomization was 0.8 months (D-VMP: 0.76; VMP: 0.82). The majority of subjects had in IgG (64%), IgA (22%), and light chain (10%) myeloma; 19% had ISS Stage I, 42% had ISS Stage II, and 38% had ISS Stage III disease at screening. Of the 616 subjects who had baseline cytogenetic data reported, 16% had a high-risk cytogenetic abnormality, as defined by del17p, t(4;14) or t(14;16) determined by fluorescence in situ hybridization (FISH) or karyotype testing. The distribution of subjects with renal or hepatic impairment at enrollment was balanced between the treatment groups, with 42% having moderate or severe renal impairment (creatinine clearance [CrCL]<60 mL/min; D-VMP: 43%; VMP: 41%) and 14% (D-VMP: 13%; VMP: 15%) having impaired hepatic function by National Cancer Institute organ dysfunction criteria.

To control for any variation in transplant ineligibility criteria among countries or regions, subjects were considered transplant ineligible if they were aged ≥65 years; if <65 years old, subjects had to have important comorbid conditions deemed likely to have a negative impact on tolerability to the HDT used in ASCT. Age was the main transplant ineligibility criterion in this study (92%).

The study population was generally consistent with subjects enrolled in other clinical trials involving subjects with newly diagnosed multiple myeloma ineligible for transplant (Benboubker L, et al. N Eng J Med. 2014; 371:906-917; Mateos M V, et al. Lancet Oncol. 2010; 11:934-941; San Miguel J F, et al. N Engl J Med. 2008; 359:906-917; hereby incorporated by reference in their entirety).

2-3 Efficacy Results

Data from the controlled Phase 3 Study demonstrate that the combination of daratumumab with standard of care background therapy, VMP, results in highly compelling and clinically meaningful outcomes for patients with newly diagnosed multiple myeloma ineligible for ASCT. In this study, treatment with D-VMP resulted in a highly statistically significant improvement in PFS compared with VMP alone. The superiority in PFS was further supported by consistent, statistically significant improvements in ORR and depth of response for D-VMP compared with VMP, including a near doubling in the CR or better rate and a more than tripling in the MRD negativity rate. Of note, efficacy results for the VMP control group were consistent with those for VMP in the VISTA study.

2-3-1 Primary Endpoint—Progression-Free Survival

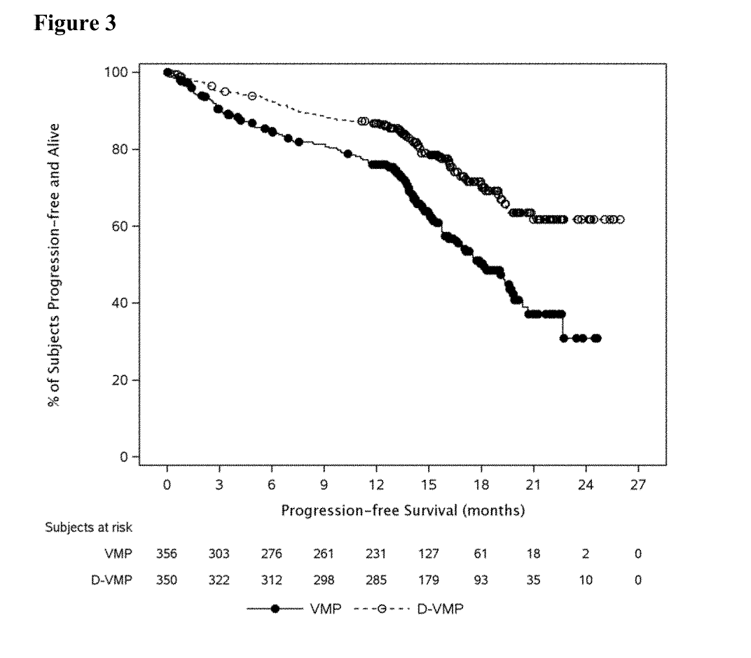

Treatment with D-VMP resulted in a 50% reduction in the risk of disease progression or death compared with VMP alone (HR=0.50; p<0.0001) (Table 1). The median PFS was not reached in the D-VMP group and was 18.1 months in the VMP group, which is consistent with the median PFS for the frontline VMP regimen used in the VISTA study. Kaplan-Meier curves for PFS are provided in FIG. 3 and show early separation of the curves favoring D-VMP shortly after randomization which persisted and continued to widen over time.

TABLE 1
Primary and Sensitivity Analyses of Progression-free Survival;
Intent-to-treat Analysis Set

| | VMP | D-VMP |
|---|---|---|
| Intent-to-treat analysis set | 356 | 350 |
| Primary analysis | | |
| Median (95% CI) (months) | 18.14 (16.53, 19.91) | NE (NE, NE) |
| Hazard ratio (95% CI) | | 0.50 (0.38, 0.65) |
| P-value | | <0.0001 |
| Sensitivity analysis | | |
| Investigator assessment | | |
| Hazard ratio (95% CI) | | 0.53 (0.40, 0.69) |
| P-value | | <0.0001 |
| Not censored for subsequent | | |
| therapy | | |
| Hazard ratio (95% CI) | | 0.50 (0.39, 0.66) |
| P-value | | <0.0001 |
| Censored for events after more | | |
| than one missed disease | | |
| assessment | | |
| Hazard ratio (95% CI) | | 0.48 (0.36, 0.64) |
| P-value | | <0.0001 |
| Un-stratified analysis | | |
| Hazard ratio (95% CI) | | 0.50 (0.39, 0.66) |
| P-value | | <0.0001 |

Key:
CI = confidence interval;
D-VMP = daratumumab + bortezomib + melphalan + prednisone;
NE = not estimable;
VMP = bortezomib + melphalan + prednisone.
Note:
The median is based on a Kaplan-Meier product-limit estimate. The hazard ratio and 95% CI for progression-free survival are based on a stratified Cox proportional hazards model unless otherwise specified. The p-value is based on a stratified log-rank test unless otherwise specified. A hazard ratio <1 indicates an advantage for the D-VMP arm.

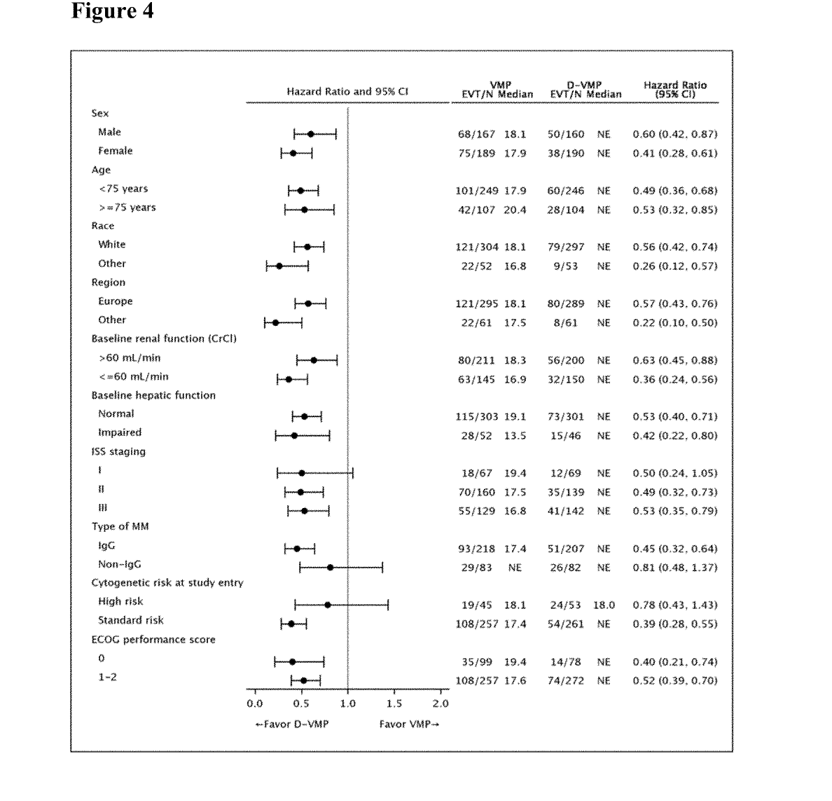

All pre-planned sensitivity analyses, including investigator-assessed PFS, demonstrated robust and consistent results with the primary analysis (Table 1). Furthermore, subgroup analyses of PFS demonstrated that the treatment effect of D-VMP over VMP was consistent across all pre-specified, clinically relevant subgroups (FIG. 4), including subjects aged 75 years or older, those with renal impairment, those with advanced-stage disease (ISS Staging III), and those with high-risk cytogenetic abnormalities.

2-2 Key and Other Secondary Endpoints

Based upon the significant findings for the primary endpoint of PFS, pre-specified hierarchical testing along with alpha spending using group sequential methods was performed for the key secondary efficacy endpoints to strongly control the family-wise type I error rate at 0.05 (2-sided). The p-values for ORR, VGPR or better rate, CR or better rate, and MRD negativity rate all crossed the O'Brien-Fleming stopping boundary of 0.0244 as pre-specified.

The administration of D-VMP resulted in statistically significant improvements relative to VMP for ORR, VGPR or better rate, CR or better rate, and MRD negativity rate. OS data were not mature at the time of the clinical cut-off. Results for the key secondary efficacy endpoints based on response assessed by the computerized algorithm are summarized in Table 2 and discussed below. Also discussed below are results for select other pre-specified secondary endpoints.

TABLE 2
Summary of Key Response-related Secondary Efficacy Endpoints;
Intent-to-treat Analysis Set

| | VMP | D-VMP |
|---|---|---|
| Intent-to-treat analysis set | 356 | 350 |
| Overall response | | |
| (sCR + CR + VGPR + PR) | | |
| n (%) | 263 (73.9%) | 318 (90.9%) |
| (95% CI) | (69.0%, 78.4%) | (87.3%, 93.7%) |
| Odds Ratio (95% CI) | | 3.55 (2.30, 5.49) |
| P-value^{a} | | <0.0001 |
| VGPR or better | | |
| (sCR + CR + VGPR) | | |
| n (%) | 177 (49.7%) | 249 (71.1%) |
| (95% CI) | (44.4%, 55.0%) | (66.1%, 75.8%) |
| Odds Ratio (95% CI) | | 2.50 (1.83, 3.41) |
| P-value^{a} | | <0.0001 |
| CR or better(sCR + CR) | | |
| n (%) | 87 (24.4%) | 149 (42.6%) |
| (95% CI) | (20.1%, 29.2%) | (37.3%, 47.9%) |
| Odds Ratio (95% CI) | | 2.31 (1.67, 3.20) |
| P-value^{a} | | <0.0001 |
| MRD negativity rate (10⁻⁵) | | |
| n (%) | 22 (6.2%) | 78 (22.3%) |
| 95% CI of MRD negativity rate | (3.9%, 9.2%) | (18.0%, 27.0%) |
| Odds ratio with 95% CI | | 4.36 (2.64, 7.21) |
| P-value^{b} | | <0.0001 |

Key:
CI = confidence interval;
CR = complete response;
D-VMP = daratumumab + bortezomib + melphalan + prednisone;
ISS = International Staging System;
MRD = minimal residual disease;
PR = partial response;
sCR = stringent complete response;
VGPR = very good partial response;
VMP = bortezomib + melphalan + prednisone.
Note:
Mantel-Haenszel estimate of the common odds ratio for stratified tables is used. The stratification factors are: ISS staging (I, II, III), region (Europe vs. Other), and age (<75 years vs. ≥75 years) as randomized. An odds ratio >1 indicates an advantage for D-VMP.
^{a}P-value from Cochran-Mantel-Haenszel chi-square test.
^{b}P-value from Fisher's exact test.

2-2-1 Response Rates

Improvements in response rates were compelling, with an ORR (based on the best confirmed response via the computerized algorithm in the ITT analysis set) of 91% for the D-VMP group, which was statistically significantly higher (odds ratio=3.55; p<0.0001) than the ORR observed in the VMP group (74%). Combining daratumumab with the standard of care VMP regimen led to a rapid improvement in the depth of response when compared with VMP alone. Within the first 12 months of treatment (ie, maximum duration of VMP treatment), improvements were seen for the D-VMP group in ORR (D-VMP: 91%; VMP: 74%), VGPR or better rate (D-VMP: 70%; VMP: 49%), and CR or better rate (D-VMP: 34%; VMP: 21%). Continued treatment with daratumumab monotherapy after discontinuation of VMP (after Cycle 9) further deepened responses as evidenced by an increased CR or better rate to 43% overall compared to 34% in the first 12 months.

The rate of VGPR or better (stringent CR+CR+VGPR) by computerized algorithm was significantly higher (p<0.0001) for the D-VMP group than for the VMP group (71% vs 50%; odds ratio=2.50). Similarly, the rate of CR or better was significantly higher (p<0.0001) and nearly doubled for the D-VMP group compared with the VMP group (43% vs 24%; odds ratio=2.31) (Table 2). The stringent CR rate was more than doubled for the D-VMP group compared with the VMP group (18% vs 7%) in the ITT population.

Results for ORR and the rate of CR or better favoring the D-VMP group over the VMP group were consistent across all pre-specified subgroups.

Results of the best confirmed response based on investigator assessment were consistent with those determined by the computerized algorithm, and showed statistically significant improvement in ORR and the rates for VGPR or better and CR or better for the D-VMP group compared with the VMP group.

2-2-2 Minimal Residual Disease

The D-VMP group demonstrated a greater incidence of MRD negativity compared with the VMP group. The MRD negativity rate at the sensitivity threshold of 10⁻⁵ was significantly higher (more than a tripling of the rate) for the D-VMP group (22%) compared with the VMP group (6%) (odds ratio=4.36; p<0.0001) (Table 2).

Subjects who achieved MRD negativity (10⁻⁵ threshold) experienced fewer (11% [11 of 100 subjects]) PFS events compared with MRD positive subjects (36% [220 of 606 subjects]).

2-2-3 Time to Response, Duration of Response

Response to treatment was rapid, occurring within the first month of treatment in both groups. Similar median times to first response (0.8 months for both groups), to VGPR or better (2.2 vs 2.8 months), and to CR or better (8.3 vs 7.5 months) were observed among responders in the D-VMP and VMP groups, respectively.

The median duration of response was not reached for the D-VMP group and was 21.3 months for the VMP group.

2-2-4 Additional Time to Event Efficacy Endpoints: Time to Disease Progression, Time to Subsequent Anti-myeloma Therapy, PFS2, and OS

Consistent with the results for PFS, there was a 59% reduction in the risk of disease progression for subjects treated with D-VMP compared with VMP. The median time to progression (TTP) as of the clinical cut-off was not reached for the D-VMP group and was 19 months for the VMP group. The improvement in TTP for subjects treated with D-VMP was also compelling (HR=0.41; p<0.0001).

The time to subsequent anti-myeloma therapy was longer for subjects in the D-VMP group compared with the VMP group (HR=0.48; p<0.0001). The median time to subsequent anti-myeloma therapy was not reached for either group.

A total of 44 subjects (13%) in the D-VMP group and 50 subjects (14%) in the VMP group had PFS2 event (PFS2 referring to the time from randomization to progression on the next line of therapy or death, whichever occurred first) (unstratified analysis: HR=0.82; p=0.3510). The median PFS2 was not reached in either group.

With a median follow-up of 16.5 months, OS data are not mature for this newly diagnosed population. Ninety-three (93) deaths were observed at the clinical cutoff date (45 subjects [13%] in the D-VMP group and 48 subjects [14%] in the VMP group). The HR was 0.92 (p=0.6691), and the median OS was not reached in either group.

2-2-5 Functional Status and Well-being

Patients with multiple myeloma have a high symptom burden and low health-related quality of life (HRQoL) throughout their disease course (Mols F, et al. Eur J Haematol. 2012; 89(4):311-319; Ramsenthaler C, et al. BMC Cancer. 2016; 16:427; hereby incorporated by reference in their entirety). A secondary objective of the Phase 3 Study was to assess patient-reported outcomes. The cancer-specific EORTC-QLQ-C30 (European Organization for Research and Treatment of Cancer Quality of Life Core Questionnaire) and generic EQ-ED-5L (EuroQoL 5-dimension Questionnaire) were included to assess subjects' functional status and well being. The combination of daratumumab with VMP did not negatively affect patient's HRQoL.

### Efficacy Conclusion

Data from the Phase 3 Study demonstrate that the combination of daratumumab with the standard of care VMP regimen results in a robust clinical benefit that was both statistically significant and clinically meaningful, when compared to VMP alone, for patients with newly diagnosed multiple myeloma who are ineligible for ASCT. Specifically, the combination of daratumumab with VMP resulted in a significant improvement in PFS, with a 50% reduction in the risk of disease progression or death, compared with background VMP. The superiority in PFS is further supported by robust improvements in key secondary endpoints which include a best overall response in 91% of subjects in the D-VMP group, and a near doubling of the CR rate and a more than tripling of the MRD negativity rate in the D-VMP group compared with the VMP group.

The totality of the data demonstrates that daratumumab significantly improves the outcomes of patients with newly diagnosed multiple myeloma who are ineligible for ASCT when combined with the standard of care background therapy, VMP.

### Example 3. Safety

The safety population for the Phase 3 Study consisted of data from 700 subjects (346 subjects received D-VMP; 354 subjects received VMP). In both treatment groups, VMP was to be administered for the first 9 cycles of treatment and then stopped, as per the VELCADE prescribing information (VELCADE® [Prescribing Information] 2017; VELCADE® [Summary of Product Characteristics] 2014). In the D-VMP group, daratumumab administration continued for the entire treatment period (ie, post Cycle 9), while subjects in the VMP group entered the Follow-up phase with no VMP treatment after Cycle 9. Thus, there is a potential for bias with respect to reporting AEs in the D-VMP group due to a longer exposure. The safety population includes all subjects who received at least 1 dose of study treatment.

3-1 Exposure to Treatment

At the time of clinical cut-off, subjects in the D-VMP group received a median of 12 treatment cycles while subjects in the VMP group received a median of 9 treatment cycles; consequently, the median duration of treatment was longer for the D-VMP group (14.7 months) than for the VMP group (12.0 months). Approximately 71% of subjects in the D-VMP group, and 5% in the VMP group, were still receiving treatment at the time of clinical cut-off (see FIG. 2). The median relative overall dose intensities were similar for the 2 treatment groups for bortezomib (D-VMP: 96%, VMP: 94%), melphalan (97% each), and prednisone equivalent (99% each).

3-2 Safety

3-2-1 All Grade and Grade 3 or 4 Adverse Events

Adverse events for daratumumab in combination with VMP demonstrate a manageable safety profile, and were consistent with the known toxicities of daratumumab and the VMP regimen. Combining daratumumab with VMP did not result in additional toxicity, with the exception of IRRs and a higher rate of infection.

The combination of daratumumab with VMP did not increase the overall frequency of treatment-emergent adverse events (TEAEs), either of all grades (D-VMP: 97%; VMP: 97%) or of Grade 3 or 4 events (D-VMP: 78%; VMP: 77%). The TEAE profile was generally similar between the 2 treatment groups, with the frequencies for the majority of the common TEAEs (ie, ≥20% in either group) balanced between the D-VMP and VMP groups. TEAEs reported in at least 10% of subjects in either group and at a ≥5.0% higher frequency in the D-VMP group compared with the VMP group, respectively, were upper respiratory tract infection (26% vs 14%), pneumonia (15% vs 5%), bronchitis (15% vs 8%), edema peripheral (18% vs 11%), cough (15% vs 8%), dyspnea (12% vs 5%), and hypertension (10% vs 3%). TEAEs reported in at least 10% of subjects in either group and at a ≥5.0% lower frequency in the D-VMP group compared with the VMP group, respectively, were anemia (28% vs 38%) and peripheral sensory neuropathy (28% vs 34%).

Common Grade 3 or 4 TEAEs in the D-VMP and VMP groups were mainly hematologic: neutropenia (40% vs 39%, respectively), thrombocytopenia (34% vs 38%, respectively), and anemia (16% vs 20%, respectively). The frequencies of the remaining Grade 3 or 4 TEAEs were well balanced across the treatment groups except for pneumonia, which occurred at a higher rate in the D-VMP group (11% vs 4% in VMP group).

3-2-2 Adverse Events Leading to Treatment Modification

Dose modifications of daratumumab typically took the form of dose delays or skipped doses; daratumumab dose reductions were not allowed. In the D-VMP group, 40% of subjects were reported to have at least 1 TEAE leading to dose modification of daratumumab, with thrombocytopenia (12%) and neutropenia (11%) being the most common TEAEs leading to daratumumab dose modification. This is generally consistent with the experience reported in clinical studies of daratumumab in relapsed or refractory multiple myeloma.

A similar proportion of subjects from the D-VMP and VMP groups were reported with a TEAE leading to dose modifications of bortezomib (D-VMP: 57%; VMP: 56%), melphalan (D-VMP: 22%; VMP: 23%), or steroids (D-VMP: 8%; VMP: 7%).

3-2-3 Adverse Events Leading to Discontinuation of Treatment

Discontinuation of all study treatments due to TEAEs was reported at a lower frequency in the D-VMP group (5%) compared with the VMP group (9%). Upper respiratory tract infection (2 subjects, 0.6%) was the only TEAE that resulted in discontinuation of study treatment in more than 1 subject in the D-VMP group. In the VMP group, peripheral sensory neuropathy (6 subjects, 1.7%), fatigue (2 subjects, 0.6%), and neuralgia (2 subjects, 0.6%) were those TEAEs leading to discontinuation of all study treatment in >1 subject. Discontinuation of daratumumab alone in the D-VMP group was also infrequent (7%).

3-3 Serious Adverse Events and Deaths

3-3-1 Serious Adverse Events

The overall incidence of serious TEAEs was higher for the D-VMP group (42%) than for the VMP group (33%). Most serious TEAEs were in the Infections and Infestations system organ class (D-VMP: 23%; VMP: 12%), consistent with the experience reported for daratumumab in clinical studies in subjects with relapsed or refractory multiple myeloma. Pneumonia was the only serious TEAE reported in at least 5% of subjects in either group, and was reported at a higher rate in the D-VMP versus the VMP group (10% vs 3%, respectively). Serious pulmonary edema was reported in 1.7% of subjects in the D-VMP group and in no subject in the VMP group.

3-3-2 Deaths

Deaths within 30 days of last study treatment dose were infrequent and balanced between the 2 treatment groups (D-VMP: 14 subjects, 4.0%; VMP: 16 subjects, 4.5%).

TEAEs with an outcome of death were also low and balanced between the treatment groups, reported for 19 subjects (5.5%) in the D-VMP group and for 19 subjects (5.4%) in the VMP group. Pneumonia and death were the only individual preferred terms with an outcome of death in more than 1 subject in the D-VMP group (2 subjects each).

3-4 Adverse Events of Special Interest

3-4-1 Infusion-Related Reactions

IRRs were determined by the investigators and identified as such in the case report forms. The incidence of IRRs (all grades) in the D-VMP group was 28%. The preferred terms, severity, and onset of IRRs in the Phase 3 Study were consistent with those previously reported following daratumumab monotherapy and combination therapy.

The most frequently reported TEAE terms (reported in ≥5% of subjects) used to describe IRRs were dyspnea (7%), chills (6%), and hypertension (5%). The majority of IRRs were mild (Grade 1 or 2), with Grade 3 IRRs reported for 4% of subjects, and only 2 subjects (0.6%) having a Grade 4 IRR.

IRRs typically occurred early. The incidence of any grade IRRs in the D-VMP group was 26% with the first infusion, 2% with the second infusion, and 4% with subsequent infusions. Only 4.0% of subjects experienced an IRR in more than 1 infusion.

IRRs were effectively managed with pre-medications, infusion interruptions, and symptomatic treatment, and did not usually result in treatment discontinuation. A total of 5 subjects (1.4%) in the D-VMP group discontinued daratumumab due to IRRs, and no IRRs had an outcome of death.

3-4-2 Cytopenia Adverse Events

Cytopenias were the most frequently reported TEAEs in both treatment groups, and the overall incidence of treatment-emergent cytopenia events was balanced for the D-VMP (73% all grades; 60% Grade 3 or 4) and VMP (76% all grades, 62% Grade 3 or 4) groups. Hematology laboratory results (see Example 3-6-1) were consistent with reported cytopenia TEAEs. Neutropenia and thrombocytopenia are included in the Warnings and Precautions section of the daratumumab product information (DARZALEX® [Prescribing Information] 2017; DARZALEX® [Summary of Prescribing Characteristics] 2016).

Neutropenia:

The frequency of neutropenia events (including events of febrile neutropenia and neutropenic infection) was similar for the D-VMP (50% all grades; 40% Grade 3 or 4) and VMP (53% all grades, 40% Grade 3 or 4) groups. No subject in either treatment group discontinued all study treatments due to a neutropenia TEAE. The frequency, severity, temporal course, and management of neutropenia events was generally consistent with that reported for daratumumab given in combination with standard background therapy in subjects with relapsed or refractory multiple myeloma.

Thrombocytopenia:

Thrombocytopenia was reported in 49% of subjects in the D-VMP group (34% Grade 3 or 4) and 54% of subjects in the VMP group (38% Grade 3 or 4). For a majority of subjects in both treatment groups who experienced thrombocytopenia TEAEs, the first onset of this event occurred in Cycle 1 (D-VMP: 138 of 169 subjects; VMP: 157 of 190 subjects). One subject (0.3%) in each treatment group discontinued all study treatment due to thrombocytopenia.

Although thrombocytopenia was reported by approximately one-half of subjects in both treatment groups, the incidence of hemorrhage was low for both groups (D-VMP: 15% all grades, 3% Grade 3 or 4; VMP: 11% all grades, 2% Grade 3 or 4). One subject in the D-VMP group had a Grade 5 hemorrhage event (hemorrhage intracranial) which was not associated with thrombocytopenia. Overall, there was no evidence suggesting a relationship between Grade 3 or 4 hemorrhage and thrombocytopenia.

Anemia:

Fewer subjects in the D-VMP group had a TEAE of anemia (28% all grades; 16% Grade 3 or 4) compared with subjects in the VMP group (38% all grades; 20% Grade 3 or 4). The lower frequency of anemia TEAEs in the D-VMP group was consistent with a larger increase in hemoglobin levels with treatment suggestive of improving anemia in the D-VMP group (see Example 3-6). The management of anemia using red blood cell transfusions was also less in the D-VMP group (21%) compared with the VMP group (26%); use of whole blood transfusions (D-VMP: 2%; VMP: 1%) or erythropoietin or anti-anemic preparations (D-VMP: 27%; VMP: 28%) was similar in the 2 groups.

Lymphopenia was reported as a TEAE in a similar proportion of subjects in the D-VMP (11%; 8% Grade 3 or 4) and VMP (10%; 6% Grade 3 or 4) groups.

3-4-3 Infections and Infestations

Patients with multiple myeloma have an increased risk of infections due to underlying disease causing hypogammaglobulinemia and immunosuppression (Terpos E, Haematologica 2015; 100:1254-1266, hereby incorporated by reference in its entirety). Consistent with this known risk, TEAEs in the Infections and Infestations system organ class were among the most frequently reported events in the Phase 3 Study, and were reported at a higher frequency in the D-VMP group (67% all grades, 23% Grade 3 or 4) than in the VMP group (48% all grades, 15% Grade 3 or 4). The majority of all infections were mild (Grade 1 or 2) and did not require hospitalization, and infections rarely resulted in discontinuation of study treatment or death.

The only Grade 3 or 4 infection TEAE that was reported at a ≥5.0% difference between treatment groups was pneumonia, which was reported in 11% of subjects in the D-VMP group and 4% of subjects in the VMP group.

Most infections, including pneumonia, resolved over time and did not result in increased rates of discontinuation of study treatments (infection leading to discontinuation: D-VMP: 3 subjects [0.9%]; VMP: 5 subjects [1.4%]). Pneumonia (any grade) resulted in discontinuation of study treatments in 1 subject (0.3%) in each treatment group.

Deaths due to infection were infrequent and balanced between groups, reported for 5 subjects (1.4%) in the D-VMP group (pneumonia [2 subjects], peritonitis, septic shock, upper respiratory tract infection [1 subject each]), and 4 subjects (1.1%) in the VMP group (septic shock, candida sepsis, pneumonia bacterial, sepsis [1 subject each]).

Opportunistic Infections:

Twelve percent (12%) of subjects in the D-VMP group and 9% of subjects in the VMP group had a treatment-emergent opportunistic infection, with 2% of subjects in both groups having a Grade 3 or 4 opportunistic infection.

Opportunistic infections included: Herpes zoster, Oral herpes, Oral candidiasis, Cytomegalovirus infection, Candida infection, Fungal skin infection, Brochopulmonary aspergillosis, Herpes simplex, Herpes zoster disseminated, Nasal herpes, Oropharyngeal candidiasis, Skin candida, Tuberculosis, Varicella zoster virus infection, Vulvovaginal candidiasis, Candida sepsis, Herpes virus infection, Oesophageal candidiasis, Onychomycosis, Pneumonia streptococcal, Tinea infection, Tuberculosis pleurisy.

The most frequently reported opportunistic infections (all grades) were herpes zoster (D-VMP: 5%; VMP: 4%) and oral candidiasis (D-VMP: 2%; VMP: 2%). Bortezomib exposure poses a known risk of herpes zoster reactivation (VELCADE® [Prescribing Information] 2017; VELCADE® [Summary or Prescribing Characteristics] 2014). A recommendation to initiate antiviral prophylaxis to prevent herpes zoster reactivation is included in the DARZALEX prescribing information (DARZALEX® [Prescribing Information] 2017; DARZALEX® [Summary of Prescribing Characteristics] 2016).

3-4-4 Second Primary Malignancies

In the Phase 3 Study, there was no increased risk of second primary malignancy (SPM) due to daratumumab treatment for patients with newly diagnosed multiple myeloma. With a median follow-up of 16.5 months, SPMs were infrequent and balanced in the D-VMP and VMP groups (8 subjects [2%] vs 9 subjects [3%], respectively). No hematologic SPMs were reported in the D-VMP group (n=2 for VMP group), and the occurrence of non-cutaneous, invasive SPMs and cutaneous, non-invasive cancers were balanced between the treatment groups. No trends in specific tumor types were identified.

3-4-5 Tumor Lysis Syndrome

The occurrence of tumor lysis syndrome (TLS) in the Phase 3 Study was infrequent and balanced for the D-VMP and VMP group (reported for 2 subjects [0.6%] in each group, all Grade 3 or higher). Of the 2 subjects with TLS in the D-VMP group, 1 had a Grade 5 event; this event was not considered to be related to daratumumab by the investigator and did not meet clinical or laboratory criteria for TLS. One subject in the VMP group had a Grade 5 TLS event.

3-4-6 Intravascular Hemolysis

Daratumumab binds to CD38 found at low levels on red blood cells and could theoretically result in hemolysis. Hemolysis was reported in only 3 subjects overall in the Phase 3 Study, including 2 in the D-VMP group and 1 in the VMP group. None of these events were Grade 3 or higher. In the D-VMP group, hemolysis was reported after packed red blood cell transfusions in 1 of the 2 subjects. For both subjects, the hemolysis event resolved, and the subjects remained on treatment without further reports of hemolysis.

3-4-7 Treatment-Emergent Interference for Blood Typing

No cases of daratumumab interference with blood typing were reported in the Phase 3 Study.

3-5 Other Adverse Events

3-5-1 Peripheral Neuropathy

Peripheral neuropathy is a common side effect of treatment with bortezomib. To improve tolerability, twice weekly dosing of bortezomib was incorporated in the first cycle of the Phase 3 Study, followed by once weekly dosing in subsequent cycles. In addition, bortezomib was administered by SC administration which has been shown to improve the safety profile and significantly lower the rates of peripheral neuropathy (Moreau P, et al. Lancet Oncol. 2011; 12(5):431-440, hereby incorporated by reference in its entirety). Subjects with peripheral neuropathy or neuropathic pain Grade 2 or higher at screening were not eligible for participation in the study.

Fewer subjects in the D-VMP group (32% all grades; 3% Grade 3 or 4) compared with the VMP group (37% all grades; 5% Grade 3 or 4) had a TEAE of peripheral neuropathy based on Medical Dictionary for Regulatory Activities (MedDRA) high level term (preferred terms, peripheral sensory neuropathy [most common], peripheral sensorimotor neuropathy, neuropathy peripheral, peripheral motor neuropathy, axonal neuropathy). Discontinuation of bortezomib due to individual peripheral neuropathy preferred terms was infrequent in both treatment groups (peripheral sensory neuropathy [D-VMP: 1.4%, VMP: 2.0%], peripheral sensory neuropathy [D-VMP: 0.6%, VMP: 0 subjects], and peripheral motor neuropathy (D-VMP and VMP: 0.3% each]).

3-6 Laboratory Evaluations

3-6-1 Hematology

Hematology laboratory values were consistent with the reported TEAEs. In both treatment groups, mean values for neutrophils, platelets, and lymphocytes followed a cyclical pattern of decreases during treatment and peaking to higher levels by Day 1 of each cycle. This pattern remained stable for the duration of the study and likely represents toxicity and recovery from bortezomib treatment as cytopenias are a known toxicity of bortezomib. For mean hemoglobin values, there was a brief peak on Day 8 of the first cycle, and values decreased to a nadir at Day 22 of the first cycle. Mean hemoglobin values gradually increased over time, reaching a peak at Cycle 4 and beginning a cyclical pattern thereafter. A greater increase in mean hemoglobin values occurred in the D-VMP group, which may reflect improving anemia by treatment.

3-6-2 Clinical Chemistry

Grade 3 or 4 biochemistry laboratory abnormalities during the study were low (<5%) in both treatment groups except for Grade 3 events of low sodium (D-VMP: 10%; VMP: 6%) and high glucose (D-VMP: 6%; VMP: 5%). Shifts in biochemistry values to Grade 3 or 4 were uncommon for both treatment groups.

3-7 Subgroup Analysis

The TEAE profile for the safety population in the Phase 3 Study was analyzed to evaluate potential differences in the safety of daratumumab in combination with VMP in subjects defined by subgroups of age, sex, race, baseline renal function (adjusted), baseline hepatic function, and geographic region. The safety profile (overall incidence of TEAEs, serious TEAEs, Grade 3 or 4 TEAEs, treatment discontinuations, and TEAEs with an outcome of death) for D-VMP compared with VMP was generally consistent across all subgroups.

While there was a general trend in increasing TEAEs with increasing age for both treatment groups, the pattern of safety results in the oldest subjects (≥75 years old) (ie, similar frequency of Grade 3 or 4 TEAEs in 2 groups; higher frequency of serious TEAEs and lower rate of discontinuation due to TEAEs in D-VMP group compared with VMP group) was consistent with that seen in the overall population. In addition, deaths due to AEs were balanced between the D-VMP and VMP groups. Together, these results suggest that D-VMP is well-tolerated in elderly subjects, including those 75 years of age or older.

3-8 Adverse Drug Reactions

Medical experts evaluated safety data using the definition of adverse drug reactions (ADRs) from the International Council for Harmonisation of Technical Requirements for Pharmaceuticals for Human Use (ICH) guideline entitled, E6: Good Clinical Practice, Consolidated Guideline (ICH, 1996).

Adverse drug reactions in the Phase 3 Study were evaluated according to the following points:
All TEAEs reported during Cycles 1 to 9 in ≥10% patients in the D-VMP group and occurring with ≥5% difference to the VMP group were considered as ADRs. However, thrombocytopenia, neutropenia, lymphopenia, and anemia were excluded from the ADR tables and were instead listed in a separate table based on hematology laboratory parameters.
Serious TEAEs reported with ≥2% difference in the D-VMP group over the VMP group were considered as ADRs.
All laboratory parameters were reviewed. No laboratory parameters had an incidence of Grade 3 or 4 values ≥10% except for hematology parameters.
Hematological laboratory parameters (thrombocytopenia, neutropenia, lymphopenia and anemia) are included as a separate table regardless of the incidence and difference between groups.
The following AE terms represent a grouping of MedDRA preferred terms to reflect more accurately the incidence of ADRs: peripheral edema, pneumonia, upper respiratory tract infection, cough, dyspnea, pulmonary edema, and hypertension.

Adverse drug reactions identified are listed in Table 3, while hematology abnormalities are summarized in Table 4.

Six subjects (2%) in the D-VMP group and no subject in the VMP group had a serious TEAE of pulmonary edema. Pulmonary edema was associated with an IRR in 1 of the 6 subjects. The serious pulmonary edema was reported as resolved in all 6 subjects, and 4 of the 6 subjects received subsequent daratumumab treatment without recurrence of the pulmonary edema. One subject permanently discontinued daratumumab but continued to receive VMP alone, and 1 subject withdrew study consent and did not receive further study treatment.

Of the 35 subjects in the D-VMP group, 16 (4.6%) had 1 or more TEAEs of hypertension associated with an IRR, which contributes to the increased frequency of hypertension in the D-VMP group.

The most frequent adverse reactions (≥20%) in the D-VMP group were infusion reactions, upper respiratory tract infection, and edema peripheral. Serious adverse reactions with at least a 2% greater incidence in the D-VMP group compared with the VMP group were pneumonia (grouped term; D-VMP: 11%; VMP: 4%), upper respiratory tract infection (grouped term; D-VMP: 5%; VMP: 1%), and pulmonary edema (grouped term; D-VMP: 2%; VMP 0%).

Infusion-Related Reactions:

Data from the Phase 3 Study were combined with other daratumumab monotherapy and combination studies to evaluate the pattern of IRRs with daratumumab. The incidence of any grade IRR among the 1,166 subjects treated with daratumumab 16 mg/kg IV was 40% with the first infusion of daratumumab, 2% with the second infusion, and 4% with subsequent infusions. Fewer than 1% of subjects had a Grade 3 IRR with the second or subsequent infusion, and Grade 4 IRRs were reported in 2 of the 1,166 (0.2%) subjects.

In this pooled population, the median time to onset of an IRR was 1.4 hours (range: 0 to 72.8 hours). The incidence of infusion modification due to IRRs was 37%. Median durations of infusion for the first, second, and subsequent infusions were 7.0, 4.2, and 3.4 hours, respectively.

Severe IRRs included bronchospasm, dyspnea, laryngeal edema, pulmonary edema, hypoxia, and hypertension. Other adverse IRRs included nasal congestion, cough, chills, throat irritation, vomiting, and nausea.

TABLE 3
Adverse Drug Reactions; Safety Population

| | D-VMP | | | VMP | | |
|---|---|---|---|---|---|---|
| | Any | | | Any | | |
| | Grade | Grade 3 | Grade 4 | Grade | Grade 3 | Grade 4 |
| Analysis set: safety | 346 | | | 354 | | |
| Infusion related reactions | 96 (27.7%) | 15 (4.3%) | 2 (0.6%) | 0 | 0 | 0 |

General disorders and administration site conditions

| | | | | | | |
|---|---|---|---|---|---|---|
| Oedema peripheral* | 72 (20.8%) | 3 (0.9%) | 1 (0.3%) | 48 (13.6%) | 2 (0.6%) | 0 |

Infections and infestations

| | | | | | | |
|---|---|---|---|---|---|---|
| Upper respiratory tract infection* | 165 (47.7%) | 19 (5.5%) | 0 | 100 (28.2%) | 9 (2.5%) | 0 |
| Pneumonia* | 57 (16.5%) | 41 (11.8%) | 1 (0.3%) | 21 (5.9%) | 17 (4.8%) | 1 (0.3%) |

Respiratory, thoracic and mediastinal disorders

| | | | | | | |
|---|---|---|---|---|---|---|
| Cough* | 55 (15.9%) | 1 (0.3%) | 0 | 30 (8.5%) | 1 (0.3%) | 0 |
| Dyspnoea* | 44 (12.7%) | 6 (1.7%) | 3 (0.9%) | 17 (4.8%) | 3 (0.8%) | 0 |
| Pulmonary oedema* | 8 (2.3%) | 3 (0.9%) | 1 (0.3%) | 1 (0.3%) | 1 (0.3%) | 0 |

Vascular disorders

| | | | | | | |
|---|---|---|---|---|---|---|
| Hypertension* | 36 (10.4%) | 13 (3.8%) | 1 (0.3%) | 11 (3.1%) | 6 (1.7%) | 0 |

Key:
D-VMP = daratumumab + bortezomib + melphalan + prednisone;
VMP = bortezomib + melphalan + prednisone.
*Indicates a grouping of preferred terms.
Note:
Infusion reaction includes terms determined by investigators to be related to infusion.
Note:
Adverse events are reported using MedDRA version 20.0.
Note:
Percentages are calculated with N as the denominator, the number of safety subjects in each treatment arm.

TABLE 4 3
Treatment-emergent Hematology Lab Abnormalities; Safety Population

| | D-VMP | | | VMP | | |
|---|---|---|---|---|---|---|
| | All Grade | Grade 3 | Grade 4 | All Grade | Grade 3 | Grade 4 |
| Analysis set: safety | 346 | | | 354 | | |
| Anemia | 161 (46.5%) | 61 (17.6%) | 0 | 177 (50.0%) | 75 (21.2%) | 0 |
| Thrombocytopenia | 305 (88.2%) | 92 (26.6%) | 39 (11.3%) | 311 (87.9%) | 91 (25.7%) | 56 (15.8%) |
| Neutropenia | 297 (85.8%) | 116 (33.5%) | 34 (9.8%) | 307 (86.7%) | 112 (31.6%) | 38 (10.7%) |
| Lymphopenia | 293 (84.7%) | 158 (45.7%) | 43 (12.4%) | 294 (83.1%) | 155 (43.8%) | 33 (9.3%) |

Key:
D-VMP = daratumumab + bortezomib + melphalan + prednisone;
VMP = bortezomib + melphalan + prednisone.
Note:
The laboratory toxicity grades are derived based on the NCI CTCAE (National Cancer Institute Common Terminology Criteria for Adverse Events) Version 4.03.
Note:
For each parameter, the percentage of subjects represents those subjects for whom the toxicity grade worsened during treatment compared to baseline; percentages are calculated with the number of safety subjects in each treatment arm. For each subject and each parameter, the worst toxicity grade is selected.

### SAFETY CONCLUSION

Daratumumab in combination with VMP was well-tolerated in patients with newly diagnosed multiple myeloma. The combination of daratumumab with VMP did not result in additional toxicity, with the exception of IRRs and a higher rate of infection.

TABLE 5
Daratumumab Sequences

| | Amino Acid Sequence | SEQ ID NO |
|---|---|---|
| VH | EVQLLESGGGLVQPGGSLRLSCAVSGFTFNSFAMSWVRQAPGKGLEWVSA | 1 |
| | ISGSGGGTYYADSVKGRFTISRDNSKNTLYLQMNSLRAEDTAVYFCAKDK | |
| | ILWFGEPVFDYWGQGTLVTVSS | |
| VL | EIVLTQSPATLSLSPGERATLSCRASQSVSSYLAWYQQKPGQAPRLLIYD | 2 |
| | ASNRATGIPARFSGSGSGTDFTLTISSLEPEDFAVYYCQQRSNWPPTFGQ | |
| | GTKVEIK | |
| HC | EVQLLESGGGLVQPGGSLRLSCAVSGFTFNSFAMSWVRQAPGKGLEWVSA | 3 |
| | ISGSGGGTYYADSVKGRFTISRDNSKNTLYLQMNSLRAEDTAVYFCAKDK | |
| | ILWFGEPVFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLGT | |
| | QTYICNVNHKPSNTKVDKRVEPKSCDKTHTCPPCPAPELLGGPSVFLFPP | |
| | KPKDTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQPRE | |
| | PQVYTLPPSREEMTKNQVSLTCLVKGFYPSDIAVEWESNGQPENNYKTTP | |
| | PVLDSDGSFFLYSKLTVDKSRWQQGNVFSCSVMHEALHNHYTQKSLSLSP | |
| | GK | |
| LC | EIVLTQSPATLSLSPGERATLSCRASQSVSSYLAWYQQKPGQAPRLLIYD | 4 |
| | ASNRATGIPARFSGSGSGTDFTLTISSLEPEDFAVYYCQQRSNWPPTFGQ | |
| | GTKVEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |

What is claimed is:

1. A method of treating multiple myeloma comprising administering a safe and effective amount of an antibody that specifically recognizes CD38, bortezomib, melphalan, and prednisone to a human subject with multiple myeloma.

2. The method of claim 1, wherein the subject has not received a prior treatment for multiple myeloma.

3. The method of claim 2, wherein the human subject is ineligible for autologous stem cell transplant therapy.

4. The method of claim 3, wherein administration of a safe and effective amount of the antibody that specifically recognizes CD38, bortezomib, melphalan, and prednisone provides an increase in progression-free survival.

5. The method of claim 4, wherein administration of a safe and effective amount of the antibody that specifically recognizes CD38, bortezomib, melphalan, and prednisone provides improved overall response rate, improved very good partial response or better rate or improved complete response rate.

6. The method of claim 5, wherein administration of a safe and effective amount of the antibody that specifically recognizes CD38, bortezomib, melphalan, and prednisone provides increased MRD negativity.

7. The method of claim 6, wherein administration of a safe and effective amount of the antibody that specifically recognizes CD38, bortezomib, melphalan, and prednisone provides reduced risk of disease progression.

8. The method of claim 7, wherein administration of a safe and effective amount of the antibody that specifically recognizes CD38, bortezomib, melphalan, and prednisone results in no increase in overall frequency of treatment emergent adverse events.

9. The method of claim 8, wherein the antibody that specifically recognizes CD38 is administered by intravenous infusion.

10. The method of claim 9, wherein the antibody that specifically recognizes CD38 is administered at a dose of 16 mg/kg.

11. The method of claim 10, wherein the antibody that specifically recognizes CD38 is administered once every week for 6 weeks, then once every 3 weeks for an additional 16 doses, then once every 4 weeks thereafter.

12. The method of claim 11, wherein the antibody that specifically recognizes CD38 is administered in a pharmaceutical composition comprising about 20 mg/mL of the antibody, about 25 mM acetic acid, about 60 mM sodium chloride, about 140 mM mannitol and about 0.04% w/v polysorbate-20 (PS-20); at pH 5.5

13. The method of claim 12, wherein the wherein the antibody that specifically recognizes CD38 is administered by subcutaneous injection.

14. The method of claim 13, wherein the antibody that specifically recognizes CD38 is administered by subcutaneous injection in a formulation of about 20 mg/mL of the antibody, about 25 mM acetic acid, about 60 mM sodium chloride, about 140 mM mannitol and about 0.04% w/v polysorbate-20 (PS-20); at pH 5.5 with about 30,000 U to 45,000 of recombinant human hyaluronidase (rhPH20) in 10 mM L-Histidine, 130 mM NaCl, 10 mM L-Methionine and 0.02% Polysorbate 80, pH 6.5.

15. The method of claim 11, wherein the antibody that specifically recognizes CD38 is administered by subcutaneous injection in a pharmaceutical composition comprising rhPH20 in an amount of from about 30,000 U to about 45,000 U, histidine at a concentration of from about 5 mM to about 15 mM, sorbitol at a concentration of from about 100 mM to about 300 mM, PS-20 at a concentration of from about 0.01% w/v to about 0.04% w/v; and methionine at a concentration of from about 1 mg/mL to about 2 mg/mL, at a pH of about 5.5.

16. The method of claim 15, wherein about 1,200 mg to about 5,000 mg of the antibody that specifically recognizes CD38 is administered by subcutaneous injection in a pharmaceutical composition comprising about 120 mg/mL of the antibody, about 10 mM Histidine, about 300 mM Sorbitol, about 1 mg/mL methionine, about 0.04% Polysorbate 20, and about 2000 U/ml rHuPH20, pH 5.6.

17. The method of claim 11, wherein bortezomib is administered by subcutaneous injection and melphalan and prednisone are administered orally.

18. The method of claim 17, wherein bortezomib is administered at a dose of 1.3 mg/m².

19. The method of claim 18, wherein bortezomib is administered twice every week in weeks 1, 2, 4, and 5 of a first 6-week cycle, then once every week in weeks 1, 2, 4, and 5 of subsequent 6-week cycles thereafter; and wherein melphalan and prednisone are administered on days 1-4 of each 6-week cycle.

20. The method of claim 19, wherein melphalan is administered at 9 mg/m² and prednisone is administered at 60 mg/m².

21. The method of claim 20, wherein melphalan and prednisone are self-administered.

22. The method of claim 21, wherein the antibody that specifically recognizes CD38 comprises the HCDR1, HCDR2, and HCDR3 as set forth in the amino acid sequence of SEQ ID NO: 1 and the LCDR1, LCDR2, and LCDR3 as set forth in the amino acid sequence of SEQ ID NO: 2.

23. The method of claim 22, wherein the antibody that specifically recognizes CD38 comprises the VH of SEQ ID NO: 1 and the VL of SEQ ID NO: 2 and/or the HC of SEQ ID NO: 3 and the LC of SEQ ID NO: 4.

24. The method of claim 23, wherein the antibody that specifically recognizes CD38 is daratumumab.

25. A method of selling an antibody that specifically recognizes CD38 comprising placing the antibody into the stream of commerce with a package insert that contains instructions for safe and effective treatment of patients with newly diagnosed multiple myeloma who are ineligible for ASCT using the antibody, bortezomib, melphalan, and prednisone.

26. The method of claim 25, wherein the antibody that specifically recognizes CD38 is daratumumab.

27. A method of selling a pharmaceutical composition comprising an antibody that specifically recognizes CD38 comprising placing the pharmaceutical composition into the stream of commerce with a package insert that contains instructions for safe and effective treatment of patients with newly diagnosed multiple myeloma who are ineligible for ASCT using the antibody, bortezomib, melphalan, and prednisone.

28. The method of claim 27, wherein the antibody that specifically recognizes CD38 is daratumumab.

29. A method of offering for sale an antibody that specifically recognizes CD38 comprising offering to place the antibody into the stream of commerce with a package insert that contains instructions for safe and effective treatment of patients with newly diagnosed multiple myeloma who are ineligible for ASCT using the antibody, bortezomib, melphalan, and prednisone.

30. The method of claim 29, wherein the antibody that specifically recognizes CD38 is daratumumab.

31. A composition comprising daratumumab for providing safe and effective treatment of human patients with newly diagnosed multiple myeloma who are ineligible for ASCT when administered in combination with bortezomib, melphalan and prednisone.


SEQUENCE LISTING (4 sequences)

SEQ ID NO: 1 - protein, 122 aa, Artificial Sequence [sequence omitted]

SEQ ID NO: 2 - protein, 107 aa, Artificial Sequence [sequence omitted]

SEQ ID NO: 3 - protein, 452 aa, Artificial Sequence
  HC of daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  LC of daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC